SEQUENCE LISTING (4 sequences)

SEQ ID NO: 1 - DNA, 20 bp, Artificial Sequence
  Synthetic Nucleic Acid Ligand
cacacacacacacacacaca

SEQ ID NO: 2 - DNA, 20 bp, Artificial Sequence
  Synthetic Nucleic Acid Ligand
tgtgtgtgtgtgtgtgtgtg

SEQ ID NO: 3 - DNA, 19 bp, Artificial Sequence
  Synthetic Nucleic Acid Ligand
gctgccgggaggctatcaa

SEQ ID NO: 4 - DNA, 41 bp, Artificial Sequence
  Synthetic Nucleic Acid Ligand
tacaaactcataggcgatccttttgatagcctcccggcagc

(19) United States
(12) Patent Application Publication
(10) Pub. No.: US 2003/0059807 A1
(43) Pub. Date: Mar. 27, 2003

(54) Microcalorimetric detection of analytes and binding events

(75) Inventors: Jeffrey Shawn Roach, Longmont, CO (US); Andreas Wolter, Hamburg (DE); Warren Roach, Colorado Springs, CO (US)

Correspondence Address: SWANSON & BRATSCHUN L.L.C., 1745 SHEA CENTER DRIVE, SUITE 330, HIGHLANDS RANCH, CO, 80129, US

(73) Assignee: PROLIGO LLC, Boulder, CO

(21) Appl. No.: 10/165,854

(22) Filed: Jun. 7, 2002

Related U.S. Application Data
(60) Provisional application No. 60/296,685, filed on Jun. 7, 2001.

(51) Int. Cl. C12Q 1/68; G01N 33/53; G01N 33/542; C12M 1/34

(52) U.S. Cl. 435/6; 435/7.9; 435/287.2

(57) ABSTRACT

The present invention comprises methods for detecting specific binding interactions through measuring the heat of binding generated when members of specific binding pairs interact with each other. The invention also comprises methods to detect analytes in a solution through measurement of the heat of binding or reaction generated from the interaction of the analytes with binding or reaction partners. In addition, the invention comprises detection devices that consist of spatially addressable arrays of thermistors, which are useful in the multi-parallel thermal analysis of samples. The analytical methods and devices described are particularly useful in the analysis of nucleic acids.

### RELATED APPLICATIONS

This application claims the benefit of U.S. Provisional Patent Application Serial No. 60/296,685, filed Jun. 7, 2001, entitled “Microcalorimetric Detection of Binding Events on Microarray and Solid Supports of Thermistors.”

### FIELD OF THE INVENTION

The present invention is related to methods and devices for the detection of binding events and for the detection of analytes in test solutions. The methods described herein are based upon measuring the heat of binding or reaction generated from specific binding pairs or from analytes and binding or reaction partners. The invention is also related to detection devices that are useful in multi-parallel thermal analysis, especially for the detection of biomolecular recognition events evidenced by thermal signatures.

### BACKGROUND OF THE INVENTION

Microarray technology takes advantage of the molecular recognition properties of biopolymers including, but not limited to, DNA, RNA or peptides. Passing a sample of unknown composition across an array of known sequences or other properties and observing sites of hybridization or binding allows the sequence or other properties of the unknown to be determined.

Recent advances in large scale genetic analysis utilize oligonucleotides assembled as multi-detection arrays by microfabrication techniques. Synthesis and methods of these arrays are described in Southern et al., U.S. Pat. No. 5,700,637; Fodor et al., U.S. Pat. No. 5,445,934; Brown et al., U.S. Pat. No. 5,807,522; Drmanac et al., U.S. Pat. No. 5,202,231 and Chee, U.S. Pat. No. 5,837,832. These patents, and all other patents and literature references cited herein, are hereby incorporated by reference in their entirety. The arrays allow hybridization reactions in which the immobilized oligonucleotides are explored by labeled probes or labeled amplicons for identification of variants or mutations such as single or multiple base substitutions. A specific application of microarray technology known as expression profiling may be used for quantitation of a nucleic acid specific sequence in an unknown sample based on the number of bound probe molecules. DNA microarray technology is used in many variations to determine the sequences present in a test sample by observing the hybridization behavior of the unknowns with a defined array of known sequence biopolymers. For example, Drmanac and Crkvenjakov, U.S. Pat. No. 6,018,041, describe a process of genetic sequencing by hybridization with an array of oligonucleotides. The patent focuses on a mathematically selected array of oligonucleotides of overlapping sequences that are predicted to be optimal for sequencing of genomic DNA.

Protein/peptide arrays are used in a like manner to detect protein/protein or other interactions, and comprise a rapidly growing segment of the microarray field. Examples of protein arrays and their application may be found in Chin et al., U.S. Pat. No. 6,197,599; Friend et al., U.S. Pat. No. 6,165,709; Friend et al., U.S. Pat. No. 6,246,830 and Friend et al., U.S. Pat. No. 6,218,122.

In microarrays used for the detection of hybridization, there are two main detection technologies. The first methodology uses fluorescent labels for detection. Using this method, either the probe molecules are labeled with fluorescent dyes prior to hybridization, or an intercalating dye that will bind only to sites where hybridization has occurred is added after the hybridization reaction has taken place. Both of these techniques require the use of expensive fluorescent dyes and sophisticated optical detection equipment, which is generally expensive and requires particularly skilled personnel to operate. Furthermore, fluorescently labeled probes are expensive, because they are prepared from specialty probes that must contain incorporated linkers for the attachment of dyes and from modified fluorescent dyes that are difficult to prepare and handle. Intercalating dyes are often hazardous and represent considerable risks in the laboratory as many dyes of this class have carcinogenic or mutagenic properties.

The second detection methodology involves mass spectral analysis of the array post hybridization to detect the presence or absence of molecular ions specific to the probe molecules, such as phosphate in DNA when bound to a PNA microarray, or a specific molecular mass of DNA indicative of a known sequence. This technique requires post-hybridization treatment of the microarray and depends on efficient ionization of the hybridization sites to ensure sufficient signal detection. This method also very sensitive with respect to the presence of salts in the sample that will compromise the analytical result and will lead to decreased sensitivity and low signal to noise ratios. Great care, requiring skilled personnel, must be exercised in the preparation of samples for analysis to achieve useful signal levels. Furthermore, the mass-spectroscopy equipment used in the analysis represents a major capital expense and will not be routinely available in a standard laboratory.

Thus, the current methods of hybridization detection on the surfaces of these chips require either 1) chemical modification of the analyte molecules (i.e. fluorescently labeling the probe molecules) prior to hybridization experiments, 2) dyeing of the hybridized molecules after the experiment through the use of intercalating dyes, or 3) mass spectral analysis post hybridization to examine the presence of ions characteristic of the probe molecules (i.e. phosphate from DNA). None of these methodologies offer real time signal generation from the hybridization events. Two of the three methodologies involve chemical labeling or modification of the probe analyte, thus introducing a degree of uncertainty regarding the efficiency and specificity of such modifications. The detection of probe molecule specific ions via mass spectrometry relies upon efficient and uniform ionization of the probe molecule, again introducing a degree of uncertainty into the analysis.

All of the above described methods also include a purification step in the analysis procedure that is usually performed by washing the microarray with buffer solutions at an elevated temperature. The washing step separates bound and unbound probe molecules to enable the specific detection of bound probe molecules. It is usually performed under the most stringent conditions that can differentiate between the bound and the unbound species. The washing step complicates the analysis procedure and adds cost. In many cases special heating equipment is necessary to perform the washing step at elevated temperatures. It is highly desirable to omit the washing step from the analysis procedure in order to simplify the procedure and to reduce the cost of the analysis. The current invention offers a convenient way to avoid washing steps in the application of microarrays.

Other detection methods known in the art rely on electrochemical signals. These methods have the disadvantage of requiring electrochemically active labels or the generation of an electroactive species upon reaction for detection. For example, Heller et al., U.S. Pat. No. 5,632,957, describe the use of an array of electrodes to electrophoretically direct DNA to specific locations, as well as, monitor the reactions occurring at specific locations through electrochemical means. Other examples of electrochemical signal detection in microarrays may be found in U.S. Pat. Nos. 6,123,189, 5,972,692 and 6,207,369.

The thermodynamics of molecular interactions is an ongoing field of study. Improved ligand binding moieties through optimization of thermodynamic properties are described in the prior art. For example, Lane et al., U.S. Pat. No. 5,593,834, describe optimizing DNA sequences for binding proteins or peptides through free energy values determined by semi-empirical thermochemical methods. In another example, Lane et al., U.S. Pat. No. 6,027,884, describe a method for providing the sequence of a single stranded nucleic acid molecule which, when hybridized to a complementary single stranded molecule, results in a double stranded (duplex) structure having a preselected value for a free energy parameter, e.g., a preselected Tm or a preselected affinity for a nucleic acid binding molecule. Thus, although thermodynamic parameters provide useful information regarding binding, neither direct nor indirect detection of free energy parameters is discussed in this prior art. Another typical example is set forth in Pantoliano et al., U.S. Pat. No. 6,020,141, which describes thermal shift assays in microplates. Specifically, the disclosed methods include measuring and ranking the affinity of different molecules (ligands, proteins, peptides) to immobilized target molecules by heating and observing the melting curves as measured by changes in physical parameters, such as absorbance. This does not involve the direct detection of hybridization based on heat.

In another example, Komatsu et al. (EP0504928A2) discuss the construction of an ultraminiaturized thermal analysis sensor, utilized to study the changes in heating rates of various materials and relate the changes in heating rates to changes in enthalpy associated with phase transitions. Although the inventors mention DNA in the abstract, no further reference is made to reducing DNA analysis to practice using their device. Cavicchi et al., U.S. Pat. No. 6,079,873, describe a micron scale differential scanning calorimeter. This invention is designed to cover a gross range of temperatures (up to 500° C.), and relies on heating samples with respect to references and measuring power differences required to maintain the same temperatures, correlating to relatively gross temperature differences. This methodology does not directly detect the interactions of the samples, but rather requires heating and analyzing the power differences as the samples change phase or undergo other physiochemical changes. This would equate to a post hybridization analysis, rather than a direct, real-time analysis. Additionally, Cavicchi et al. do not make any claims regarding analysis of nucleic acids, oligonucleotides or other biological samples. For these reasons, this technology does not overlap with the present device.

Recent developments in micromachining and microfabrication technology have allowed the miniaturization of sensor devices. For example, Cozzette et al., U.S. Pat. No. 5,554,339, describe the microfabrication of electronic devices useful for the analysis of biologically significant analytes. Cozzette et al., report that the devices may be useful in a large number of assays, however, the assay must generate an electroactive species in order to be detected. For example, Cozzette et al. describe the detection of nucleic acid oligomers in a hybridization reaction, detectable by a labeled oligomer complementary to a non-interfering region of the analyte of interest, or by an antibody to the hybridized product.

Another example of microfabrication is described by Quate et al., U.S. Pat. No. 6,203,983. Quate et al., describe the detection of a physical or chemical change based on the deflection of chemically functionalized micromechanical detectors. Specifically, the micromechanical detectors are cantilevers, and their deflection is measured by the change in position of reflected laser beams focused on the individual cantilever. One of the interactions described by Quate et al., is the measurement of oligonucleotide hybridization. When a nucleic acid complementary to one on the solid support comes in contact with the surface of the support, mechanical stress is induced on the cantilever, which causes the cantilever to deflect. Quate et al. indicate that the physical or chemical change can also be in the form of a heat reaction, which similarly causes the cantilever to deflect or bend where the cantilever is made of two materials, i.e., is a bimorph. Quate et al. do not describe the direct detection of the heat of reaction of oligonucleotide hybridization.

Other advances utilizing miniaturized sensing devices have occurred in the field of enzyme analysis. Connolly and Sutherland (2000) Angew. Chem. Int. Ed. 39(23):4268-4271, report that an array of thermistors can be used for chemical and biochemical catalyst screening. The array was a standard 8×12 array format, i.e., a 96-well plate. Xie et al. (1995) Analyst 120:155-160, report a similar method for the simultaneous analysis of a multi-analyte mixture employing a thermal microbiosensor. Specifically, two independent enzyme reactions were detected utilizing five thin-film thermistors located along a single microchannel. The enzymes were immobilized on agarose beads, which were sequentially packed into distinct regions of the microchannel.

To date, the use of thermistors to directly detect DNA hybridization in larger arrays, such as a 96-well plate format, or in a more miniaturized sensor, has not been reported. Lieberman, U.S. Pat. No. 6,193,413 B1, discloses a device for “an improved calorimeter for determining thermodynamic properties of chemical and biological reactions” in which the inventor describes a device incorporating an array of thermistors incorporated in to a solid state electronic device, which is in turn incorporated into an environmentally controlled chamber. Specifically, the inventor discusses measuring heat capacities and enthalpies of protein/protein, protein/peptide, and protein/DNA interactions through the use of thermistors built into an array. The inventor does not report reducing this to practice, nor does he describe monitoring DNA/DNA interactions or monitoring real time enzymatic amplification reactions, such as the polymerase chain reaction.

### SUMMARY OF THE INVENTION

The present invention describes a novel method for detecting binding between a first member of a specific binding pair and a second member of said specific binding pair. The method comprises providing a first member of the specific binding pair within a detection device, wherein said first member of the specific binding pair is associated with spatially discrete locations with respect to the detection device, contacting the detection device with a sample suspected of containing a second member of the specific binding pair, and detecting the binding between said binding pair by thermal analysis. The detection device comprises an array of thermistors and said first member of the specific binding pair is closely associated with a unique thermistor. The means of association of the first member of the binding pair with the thermistor includes, either covalent or non-covalent attachment, or spatial localization in the form of a solution that is in close proximity to the thermistors of the detection device, as in a nanovolume well. In this embodiment the binding between members of specific binding pairs can be detected in parallel for as many binding pairs as thermistors are built into the array. In one embodiment, the binding event is hybridization between complementary nucleic acids. In other embodiments the binding event includes, but is not limited to, protein/protein, peptide/peptide, protein/peptide, antigen/antibody and protein/DNA interactions.

The present invention also describes a method for detecting an analyte or a plurality different analytes in a solution. The method comprises providing one or more binding or reaction partners to said analytes within a detection device, wherein said binding or reaction partners to the analyte or analytes are associated with spatially discrete locations with respect to the detection device, contacting the detection device with a sample suspected of containing the analyte or analytes, and detecting the binding or reaction between said analytes and their binding or reaction partners by thermal analysis. The detection device is comprised of an array of thermistors and each of the binding or reaction partners to the analytes are closely associated with a unique thermistor. The means of association of the binding pair or reaction partners with the thermistor includes, either covalent or non-covalent attachment, or spatial localization in the form of a solution that is in close proximity to the thermistors of the detection device, as in a nanovolume well. In this embodiment the binding or reaction between the analyte and the binding or reaction partners can be detected in parallel for as many analytes as thermistors are built into the array. In one embodiment, the binding event is hybridization between complementary nucleic acids. In other embodiments the binding event includes, but is not limited to, protein/protein, peptide/peptide, protein/peptide, antigen/antibody and protein/DNA interactions. In a particularly preferred embodiment the reaction between the analytes and their reaction partners is a nucleic acid amplification reaction.

The present invention also provides detection devices comprising addressable arrays of thermistors that are closely associated to either a first member of a specific binding pair or to a binding or reaction partner or partners to an analyte or a plurality of different analytes. The compounds associated with the thermistors are of known composition, sequence, and/or other property or properties of interest and allow the deduction of such properties from the second member of the specific binding pair or analyte upon interaction with the second member of the specific binding pair or analyte and from the heat generated by this event. In one embodiment the addressable array of thermistors is integrated into a microelectronic chip on a silicon wafer, in a second embodiment the addressable array of thermistors is built with reservoirs that encompass the thermistors, e.g. by integration of the thermistors in a multi-well microtitre plate.

### BRIEF DESCRIPTION OF THE FIGURES

FIG. 1A illustrates a biothermal chip comprised of a microarray of individually addressable thermistors upon which probe molecules have been deposited.

FIG. 1B illustrates the process of detection utilizing the heat generated upon hybridization between the probe and target molecules.

FIG. 2 illustrates the process of heat generation on a site or spot of the microarray by chemical ligation.

FIG. 3 illustrates the process of heat generation on a site or spot of the microarray by a chain extension reaction.

FIG. 4 illustrates the process of heat generation on a site or spot of the microarray by an amplification reaction that involves a mismatch repair enzyme.

FIG. 5A illustrates a prototype biosensor plate modified to house the NTC thermistors.

FIG. 5B illustrates an expanded view of 5A showing one array of NTC thermistors connected in a bridge circuit configuration.

FIG. 5C illustrates the electronic diagram of the bridge circuit shown in 5B with outputs going to an amplifier for signal capture.

FIG. 6 illustrates an envisioned thermistor array incorporated into an integrated circuit in a solid support such as glass with probe molecules covalently attached to the thermistor sites.

FIG. 7 illustrates schematically a proposed physical stack design of the integrated circuitry for an individual cell site within a thermistor microarray.

FIG. 8A illustrates the proposed final IC block diagram for the thermistor array shown in FIGS. 6 and 7, incorporating all necessary support electronics for control and data handling.

FIG. 8B illustrates the electronic diagram for an individual measurement cell site within a thermistor array element as shown in FIGS. 7 and FIG. 8A, elements 801, 802 and 803.

FIG. 9 illustrates the injector used in the Examples.

FIG. 9A is a top cut-away view and

FIG. 9B is a side view.

FIG. 10 illustrates a side view of the injector plus biosensor plate.

FIG. 11 illustrates an averaged signal difference acquired from measuring the heats of hybridization of DNA to DNA, perfect complements, and subtracting out the blank injection signals as described in Example 2.

FIG. 12 illustrates the signals acquired from a Buffer Blank Injection, Primer Extension Reaction and the difference between the two signals. Note the close agreement on the initial section of the curve, indicating good reproducibility, and the strong signal beginning 15 seconds into the experiment as the reaction begins to generate heat compared to the blank.

FIG. 13 illustrates averaged, representative results from the DNA mismatch experiments described in Example 2, showing the signals acquired from the perfect match, single DNA base mismatch and blank experiments.

### DETAILED DESCRIPTION OF THE PREFERRED EMBODIMENTS

The present invention describes a novel method of detecting the formation of a specific binding pair by detecting the heat generated upon formation of the specific binding pair. The present invention also describes a novel method of detection of an analyte or plurality of different analytes in a solution by detecting the heat generated upon the binding or reaction of the analyte or analytes with a provided binding or reaction partner. Furthermore, the present invention provides detection devices based on arrays of thermistors that are useful for detecting binding or reaction events in a highly parallel manner through thermal analysis.

Certain terms used to describe the invention herein are defined as follows.

It is to be noted that the term “a” or “an” entity refers to one or more of that entity; for example, a specific binding pair refers to one or more specific binding pairs. As such, the terms “a” (or “an”), “one or more” and “at least one” are used interchangeably herein.

A “sample” refers herein to any mixture that contains a plurality of molecules, the identity of at least some of which can be detected using the method of this invention. This includes, but is not limited to, bodily fluids as mixtures of macromolecules obtained from an organism, such as blood plasma, urine, saliva, and any other fluid obtainable from an organism, and any sample for environmental and toxicology testing such as contaminated water and industrial effluent.

The term “analyte” refers to a molecule that is to be measured or detected using the method of this invention. Preferably the analyte is selected from the group including, but not limited to, an antibody; an antigen, including molecules capable of being recognized by antibodies such as haptens, proteins (including polypeptides and peptides), lipids, sugars, nucleic acids or drugs; and/or a ligand or a receptor. As used herein the term “target molecule” is used interchangeably with the term analyte.

“Specific binding pairs” include, but are not limited to, complementary nucleic acids, antibody/antigen, ligand/receptor, enzyme/substrate, aptamer/target, and the like. Specific binding pairs may also refer to a chimeric molecule of any of the above components joined together through a plurality of chemical linkages, and a specific binding partner to at least one of the components.

A “binding or reaction partner” is used for a molecular entity that specifically binds to or reacts with an analyte. A binding or reaction partner could constitute the second member of a specific binding pair, as defined herein, with the first member of the binding pair being represented by the analyte. Binding or reaction partners also include any specific binding pair, where one or each of the partners in the specific binding pair are derivatized with a moiety capable of undergoing a covalent reaction when brought into proximity with the second member of the specific binding pair.

“Nucleic acid” means either DNA, RNA, single-stranded or double-stranded and any chemical modifications thereof, such as PNA and LNA. Nucleic acids can be of any size and are preferably oligonucleotides. Modifications include, but are not limited to, those which provide other chemical groups that incorporate additional charge, polarizability, hydrogen bonding, electrostatic interaction, and fluxionality to the individual nucleic acid bases or to the nucleic acid as a whole. Such modifications include, but are not limited to, modified bases such as 2′-position sugar modifications, 5-position pyrimidine modifications, 8-position purine modifications, modifications at cytosine exocyclic amines, substitution of 5-bromo-uracil; backbone modifications, methylations, unusual base-pairing combinations such as the isobases isocytidine and isoguanidine and the like. Modifications can also include 3′ and 5′ modifications such as capping. Virtually any modification of the nucleic acid is contemplated by this invention.

As used herein, “complementary” refers to nucleic acids (such as DNA or RNA) which may form double-stranded structures via hydrogen bonds between nucleotide bases (adenine and thymine, guanine and cytosine) from opposite strands, e.g., are capable of Watson-Crick base-pairing. The complementary nucleic acids may be of the same type, such as in a DNA/DNA duplex, or may be of different types, forming a hybrid duplex, including DNA/RNA, DNA/LNA, DNA/siRNA and DNA/PNA, for example. Many types of backbone-, sugar-, and base-modified nucleic acids are known to those skilled in the art, and are encompassed within this invention.

“Antibodies” can include anti-sera containing antibodies or antibodies that have been purified to varying degrees. Antibodies include functional equivalents such as antibody fragments and genetically-engineered antibodies, including single chain antibodies that are capable of selectively binding to at least one of the epitopes of the target. Antibodies that may be used in the present invention also include chimeric antibodies that can bind to more than one epitope.

A “solid support” is any microfabricated solid surface to which molecules may be attached through either covalent or non-covalent bonds, or spatial localization in the form of a solution that is in close proximity, but not attached to the solid support as in a nanovolume well. Solid supports include, but are not limited to, Langmuir-Bodgett films, functionalized glass, germanium, silicon, silicon carbide, PTFE, polystyrene, gallium arsenide, gold and silver. Any other material known in the art that is capable of having functional groups such as amino, carboxyl, thiol or hydroxyl incorporated on its surface, is contemplated. This includes planar surfaces, and also spherical surfaces. As used herein the solid support is either a part of the thermistor or is itself the thermistor.

The term “detection device” as used herein means a device that has been engineered to contain a plurality of thermistors that are spatially addressable and that allow the evaluation of signals generated from the incorporated thermistors. In a preferred embodiment the thermistors of a detection device are negative temperature coefficient (NTC) thermistors. Detection devices can be constructed from a plurality of materials known to those of skill in the art. They typically contain construction materials such as, a housing and electronic materials to control their components. In one embodiment the addressable array of thermistors is integrated into a microelectronic chip on a silicon wafer, referred to herein as a “biothermal sensor chip.” In a second embodiment the addressable array of thermistors is built with reservoirs that encompass the thermistors, e.g. by integration of the thermistors in a multi-well microtitre plate, referred to herein as a “biothermal sensor plate.” A detection device prepared from a 384-well plate is described in Example 1.

As used herein, a “thermal event” refers to the heat of molecular interaction resulting from the specific interaction between two or more molecules, including but not limited to non-covalent binding interactions and covalent reactions between two or more molecules.

In one embodiment the present invention describes a novel method for detecting binding between a first member of specific binding pair and a second members of said specific binding pair. The method comprises providing a first member of the specific binding pair within a detection device, wherein said first member of the specific binding pair is associated with spatially discrete locations with respect to the detection device, contacting the detection device with a sample suspected of containing the second member of the specific binding pair and detecting the binding between said binding pair by thermal analysis. The detection device comprises an array of thermistors and the first member of the specific binding pair is closely associated with a unique thermistor. The means of association of the first member of the binding pair with the thermistor includes, either covalent or non-covalent attachment, or spatial localization in the form of a solution that is in close proximity, but not attached to the thermistor, as in a nanovolume well. As the first member of the specific binding pair finds its specific binding partner, that is spatially resolved in the detection device, a distinct, measurable amount of heat is generated, which is measured as a small change in temperature at the point of interaction. The thermistors that are located at the points of the formation of the specific binding pairs will generate signals as the temperature increases, thus providing a real time, digital profile of the binding events as they occur in the detection device. By spatially resolving the thermistor signals, the sites of interaction and thus the formation of the specific binding pairs can be determined.

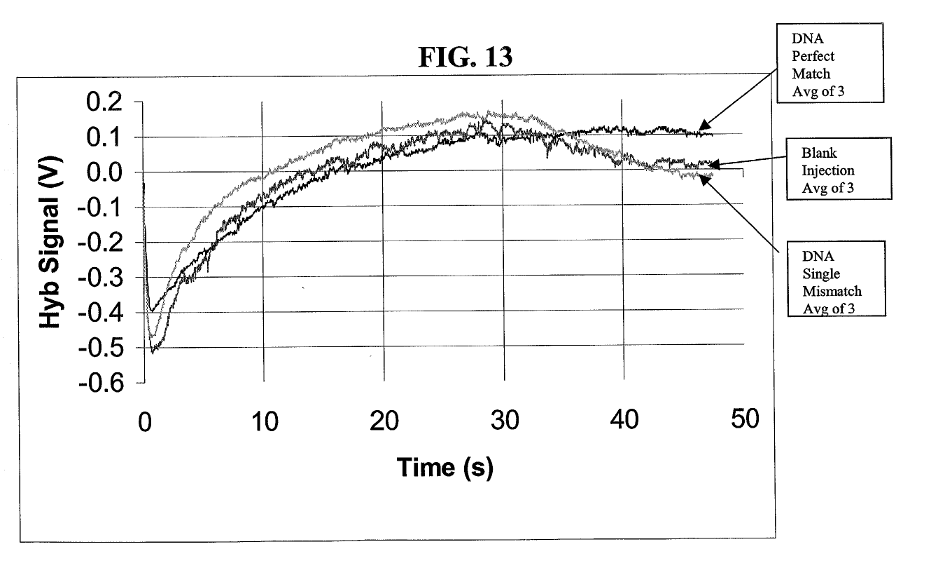

In a preferred embodiment of the present invention the members of the specific binding pairs are nucleic acids and the binding event is hybridization between complementary nucleic acids. In the preferred embodiment, the hybridization event occurs between perfectly complementary binding partners, or partners that have one or more noncomplementary (mismatch) members within the sequence as demonstrated in Example 2 and FIG. 13. By comparing and differentiating between the different levels of heat generated between perfectly complementary and noncomplementary binding partners, the invention may be used to detect sequence variations such as single nucleotide polymorphisms (SNPs), splice variants or other sequence differentiations as known in the art. In other embodiments the binding event includes, but is not limited to, protein/protein, peptide/peptide, protein/peptide, antigen/antibody and protein/DNA interactions.

The detection of specific binding can be performed in parallel for as many specific binding pairs as there are thermistors incorporated in the detection device. In this embodiment the detection device is contacted with a plurality of the first members of specific binding pairs and the thermal analysis of the formation of the specific binding pairs is carried out simultaneously.

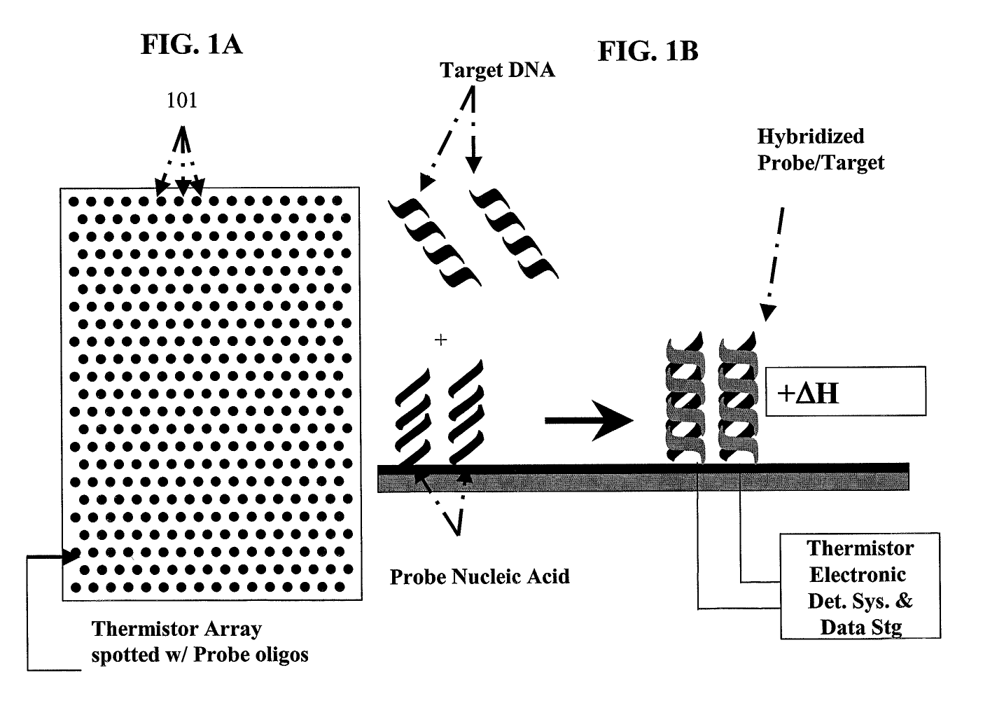

The principle of the detection of binding between members of a specific binding pair is illustrated in FIG. 1. FIG. 1A displays an array of individually addressable thermistors 101 and FIG. 1B demonstrates the generation of heat by the binding of two complimentary nucleic acids. With reference to FIG. 1B, the probe nucleic acid molecules, which are attached to individual addressable thermistors on the detection device, are brought into contact with the target DNA. The probe nucleic acid then hybridizes with the target nucleic acid generating heat in the process. The generation of heat from the formation of this specific binding pair is detected by the thermistor that produces a signal upon the hybridization of the two nucleic acids. The signal is amplified electronically and stored in a data processing unit for further evaluation.

The present invention also describes a method for detecting an analyte or a plurality different analytes in a solution. The method comprises providing one or more binding or reaction partners to said analytes within a detection device, wherein each of the said binding or reaction partners to the analytes are associated with spatially discrete locations with respect to the detection device, contacting the detection device with a sample suspected of containing the analytes, and detecting the binding or reaction between said analytes and their binding or reaction partners by thermal analysis. The detection device is comprised of an array of thermistors and each of the binding or reaction partners to the analytes are closely associated with a unique thermistor. The means of association of the binding pair or reaction partners with the thermistor includes, either covalent or non-covalent attachment, or spatial localization in the form of a solution that is in close proximity, but not attached to the thermistor, as in a nanovolume well. As the analytes find their specific binding or reaction partners, which are spatially resolved in the detection device, they generate distinct, measurable amounts of heat, measured as small changes in temperature at the point of interaction. The thermistors that are located at the points of the formation of the binding or reaction products will generate signals as the temperature increases, thus providing a real time, digital profile of the binding or reaction events as they occur in the detection device. By spatially resolving the thermistor signals, the sites of interaction and thus the presence of specific analytes can be determined.

In a preferred embodiment of the present invention the analytes and their binding or reaction partners are nucleic acids and the binding event is hybridization between complementary nucleic acids. In other embodiments the binding event includes, but is not limited to, protein/protein, peptide/peptide, protein/peptide, antigen/antibody and protein/DNA interactions.

The detection of analytes can be performed in parallel for as many analytes as there are thermistors incorporated in the detection device. In this embodiment, the detection device is contacted with a plurality of analytes and the thermal analysis of the formation of the specific binding or reaction products is carried out simultaneously.

In one embodiment of the present invention, the binding or reaction partners of analytes carry reactive moieties, which, when brought in contact with the analyte, chemically react to release heat, which is then detected by the thermistors associated with the binding or reaction partners. The reactive moieties are selected from the group including, but are not limited to, functional groups chosen to maximize the heat of reaction. The reaction between the analytes and their reaction partners may also require a catalyst to proceed. The catalyst may be either present in the sample or may be added during the process of analyzing the sample.

In another embodiment, two molecules jointly provide the binding or reaction partner of an analyte. At least one of the two binding or reaction partners of the analyte is closely associated with a unique thermistor in the detection device and the binding or reaction between the analyte and the two binding or reaction partners is detected by the release of heat from the binding or reaction event. In a preferred embodiment, the binding or reaction of the two molecules can only be detected if the two molecules and the analyte form a ternary complex. This embodiment provides the particular advantage of increased specificity, because detection is based on the formation of a ternary complex rather than a binary complex, which can be expected to form in a less specific manner.

In a particular embodiment of the present invention, one of the two molecules that jointly provide the binding or reaction partner of an analyte contains a reactive moiety which may chemically react with a second reactive moiety that is present in the analyte, if and only if, the two binding or reaction partners have formed a ternary complex with the analyte. The reactive moieties present in one of the two binding or reaction partners and the analyte may include, but are not limited to, functional groups chosen to maximize the heat of reaction. The reaction between the analyte and its reaction partners may also require a catalyst to proceed. The catalyst may be either present in the sample or may be added during the process of analyzing the sample. In one embodiment the analyte is a nucleic acid and the two binding or reaction partners of the analyte are nucleic acids that can form a ternary complex with the analyte through hybridization.

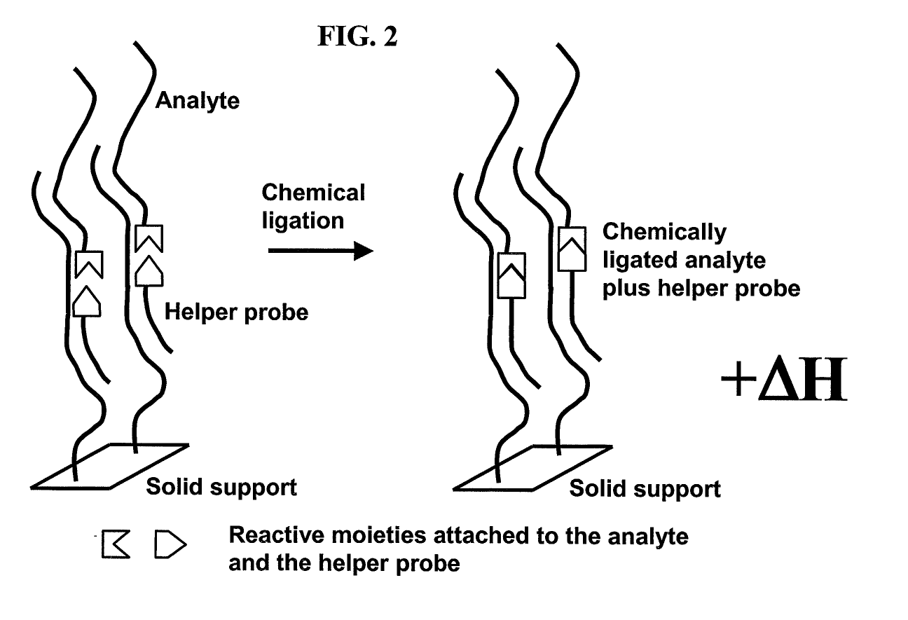

This embodiment of the invention is illustrated generally FIG. 2 using a chemical ligation as an example. With reference to FIG. 2, the analyte is a nucleic acid that carries a reactive moiety. One of the two molecules that represent the binding or reaction partners of the analyte is a second nucleic acid that is complementary to the analyte and closely associated with a thermistor. In the example, this nucleic acid is covalently attached to a solid support, which may be the thermistor itself or which may be in close contact with the thermistor. The second of the two molecules that represent the binding or reaction partners of the analyte is also a nucleic acid, which carries a reactive moiety and which is complimentary to the first of the two molecules that represent the binding or reaction partners of the analyte. Upon formation of a ternary complex between the three nucleic acids the reactive moieties of the analyte and the second binding or reaction partner of the analyte react chemically. This reaction results in the release of heat (heat of reaction), which is monitored by the thermistor. In this case, the detection relies on the chemical reaction between two functional groups of nucleic acids that are hybridized in close proximity to another complimentary nucleic acid. This process is known as the template directed chemical ligation of nucleic acids and many examples of such chemical ligations are documented. Examples include, but are not limited to the reaction of 3′-thiophosphates with 5′-iodo groups as described in Xu et al. (2000) Nat. Biotechnol. 19:148-52, and the reaction of 3′-thiophosphates with 5′-bromoacetyl groups, as described by Gryaznov et al. (1994) Nucleic Acids Res. 22:2366-2369, each of which is incorporated herein by reference in its entirety. In another embodiment of the invention, the template directed ligation reaction may be carried out in the presence of a catalyst, e.g. an enzyme that promotes the reaction. A suitable catalyst in this particular embodiment is a DNA ligase that joins a 3′—OH group of one of the nucleic acids with a 5′-phosphate of the other reaction partner.

In another embodiment of the invention, the analyte and its binding or reaction partner form a complex on the solid support that is a prerequisite for a secondary reaction to take place with the complex. The secondary reaction may or may not involve other reactants that are added to the sample. The other reactants are capable of performing the secondary reaction if and only if the specific complex between the analyte and its binding or reaction partner has formed. The secondary reaction is accompanied by a heat of reaction detected by a thermistor that is closely associated with the binding or reaction partner of the analyte. The secondary reaction may optionally require a catalyst to proceed and can be chosen from various chemical and enzymatic reactions, including but not limited to polymerizations that can be selected to provide an optimal amount of heat for detection. In one aspect of this particular embodiment, the two members of the specific binding pair are nucleic acids and the secondary reaction is a reaction catalyzed by an enzyme, such as a primer extension reaction with nucleoside triphosphates catalyzed by a DNA polymerase or an enzymatic cleavage reaction catalyzed by a sequence specific endonuclease or by an enzyme from the family of mismatch repair enzymes. In a particularly preferred aspect, the secondary reaction is a polymerization, such as a primer extension reaction, which generates heat from a multitude of monomer additions rather than from a single reaction.

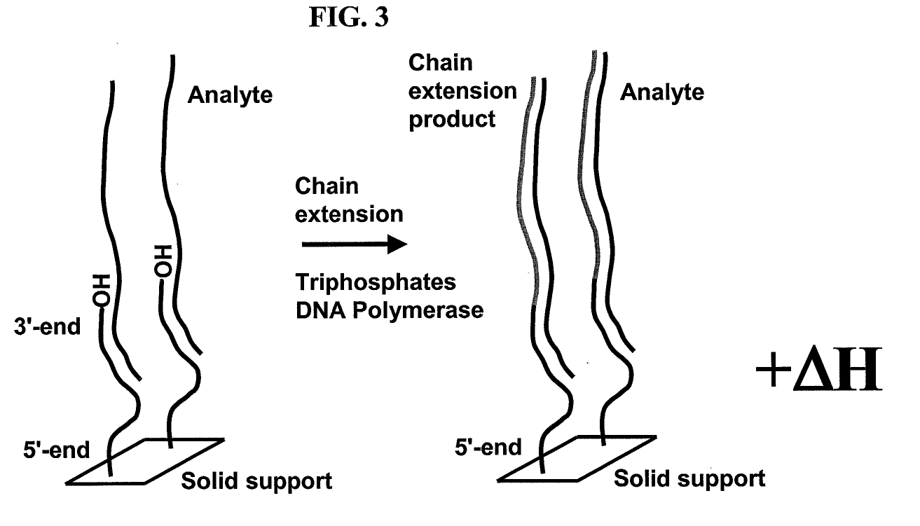

This embodiment of the invention is illustrated in FIG. 3 using a chain extension reaction for purposes of illustration. With reference to FIG. 3, the binding or reaction partner of the analyte is a nucleic acid attached to a solid support, which may be the thermistor itself or which may be in close contact with the thermistor, and the analyte is also a nucleic acid. The attachment of the binding or reaction partner of the analyte to the solid support is performed at the 5′-terminus of the nucleic acid. In this particular aspect a primer extension reaction could be performed as a secondary reaction on the support because the nucleic acid attached to the solid support displays a free 3′—OH group for the enzymatic polymerization process. Once the second member of the specific binding pair is hybridized, a chain extension reaction takes place on the support in the presence of nucleoside triphosphates and a DNA polymerase The reaction is detected through the generation of heat by the thermistor that is associated with the solid support. Such primer extension reactions are well documented in the literature, as exemplified in Erdogan et al. (2001) Nucleic Acids Res. 29:e36, which is incorporated herein by reference in its entirety. Additionally, the heat of the primer extension reaction may be amplified by use of a secondary enzyme, such as pyrophosphatase, to enhance the heat generated upon the extension of a primer through the incorporation of a nucleoside triphosphates as described in Example 2.

In yet another embodiment, the analyte and its binding or reaction partner form a complex on the solid support that is a prerequisite for an amplification reaction. An amplification reaction in the context of the invention refers to a reaction that is performed a multitude of times, if and only if, the complex between the two members of the specific binding pair is formed. The amplification reaction may or may not involve other reactants that are added to the sample. The amplification reaction is accompanied by a heat of reaction detected by the thermistor that is associated closely with the binding or reaction partner. The amplification reaction may optionally require a catalyst to proceed and can be selected to provide an optimal amount of heat for detection. In one aspect of this particular embodiment, the two members of the specific binding pair are nucleic acids and the amplification reaction is a reaction catalyzed by an enzyme, such as a cleavage reaction catalyzed by a sequence specific endonuclease or by an enzyme from the family of mismatch repair enzymes.

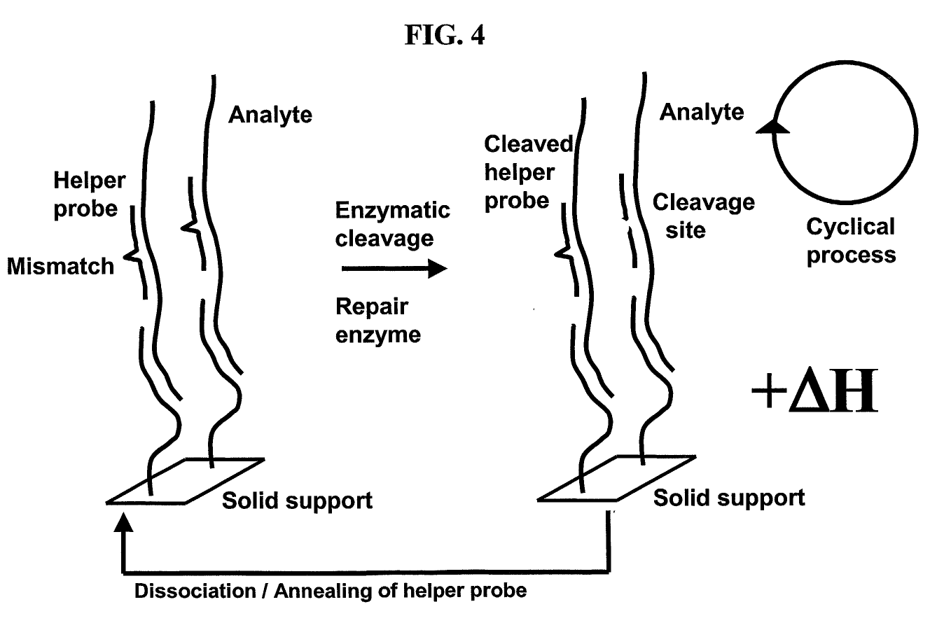

An example of this embodiment is illustrated in FIG. 4. With reference to FIG. 4, the binding or reaction partner of the analyte is a nucleic acid that is attached to a solid support, which may be the thermistor itself or which may be in close contact with the thermistor. The analyte is also a nucleic acid, which hybridizes with its binding or reaction partner on the solid support to provide a DNA duplex with a sequence overhang. After the hybridization is performed a helper probe is added that hybridizes to the sequence overhang in a manner that provides a mismatched DNA duplex comprised of the helper probe and the complex formed from the analyte and its binding or reaction partner. A repair enzyme, such as E. coli endonuclease V, then cleaves the shorter component of the mismatched duplex, i.e. the helper probe, at the mismatch site. The use of E. coli endonuclease V and other mismatch repair enzymes to detect mismatched sites in duplex DNA is described in Bazar et al. (1999) Electrophoresis 20:1141-1148 and Chirkijian et al., U.S. Pat. No. 5,656,430, each of which is incorporated by reference herein in its entirety. Upon cleavage, the nicked duplex becomes labile and the cleavage fragments of the helper probe dissociate from the complex allowing another helper probe molecule to hybridize, and subsequently be cleaved by the endonuclease. The cleavage-dissociation-hybridization cycle then continues providing an amplification reaction with an associated heat of reaction that is detected by the sensing device beneath the spot of the solid support.

There are numerous other ways or methods to generate a heat of reaction between an analyte and a binding or reaction partner, which may readily be recognized by those skilled in the art, and the examples provided should not be interpreted as to limit the way or method to generate such a heat of reaction in the context of the present invention. Instead, all exothermic reactions that can be accomplished in a selective manner between an analyte and its binding or reaction partner can be applied in the context of the invention to generate a heat of reaction that can be used to detect the analyte with a thermistor that is closely associated to the binding or reaction partner of the analyte.

The present invention also provides detection devices that are useful to detect binding between members of specific binding pairs and to detect analytes in a solution through the application of binding or reaction partners of the analytes. The detection devices of the invention are comprised of arrays of thermistors, which are spatially addressable and which are closely associated with a member of a specific binding pair or a binding or reaction partner of an analyte. As used herein, the member of a specific binding pair and/or the binding or reaction partner of an analyte that is closely associated with a specific thermistor in the detection device is referred as a “probe” in order to simplify the description of the detection devices.

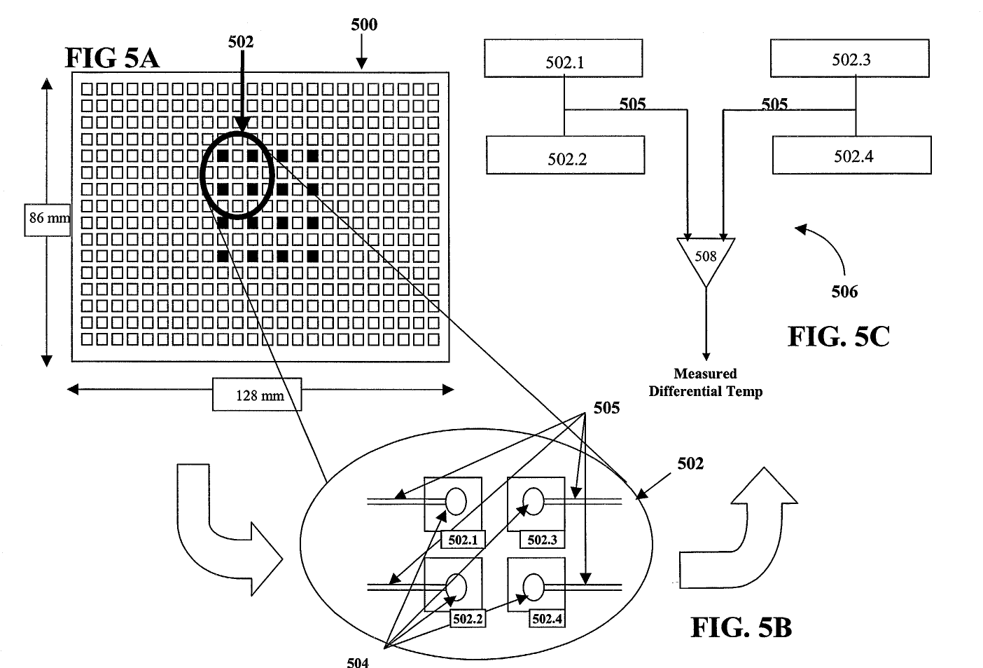

In one embodiment the array of thermistors allows the spatial resolution of probes through reservoirs that are physically separated from each other. In this embodiment, each reservoir is equipped with a thermistor and a probe that is closely associated with the thermistor. The probe could either be provided in a solution, which is in close contact with the thermistor, or could be covalently attached to a solid support, which may be the thermistor itself or may be in close contact with the thermistor. This embodiment of the invention is exemplified in FIG. 5, which displays a 384-well microtitre plate with NTC thermistors 504 incorporated into all of the wells of the plate or alternatively, in a limited number of the wells of the plate (FIG. 5A). With reference to FIG. 5, holes were drilled in the bottoms of the wells to diameters slightly smaller than the diameter of the NTC thermistors 504. Arrays of four thermistors (2×2 pattern) 502 were then incorporated into the modified plate to create four arrays with four members each 502.1, 502.2, 502.3, 502.4. FIG. 5B illustrates an expanded view of 1 array. With reference to FIG. 5B, each NTC thermistor 504 was sealed into the bottom of the individual well of the array using epoxy. Using the leads 505 from the thermistors the individual NTC elements of each array were wired together 505 to form a standard bridge circuit as illustrated in FIG. 5C. The four bridge circuits were then interfaced to a control board with standard OPAMP electronics 506, including amplification (gain) settings of 10, 100 and 1000. The measured, amplified signal was collected as the output 508 from the bridge circuit and stored in the PC.

The thermistors can be attached to the bottom of the wells, or alternatively can replace the bottom of the wells, if tightly sealed to the walls of the wells. The probe can either be added as a solution to a well of the plate or can be covalently attached to the well-plate. Covalent attachment of the probe is preferentially performed directly on the surface of the thermistor in order to maximize the sensitivity of the device. The probes may be the same in each well or may be different in some wells or may be different for every well of the plate that carries a thermistor. In this embodiment the analyte is applied to all reservoirs of the detection device simultaneously, which is conveniently achieved through robotic devices that transport a multitude of liquid samples simultaneously and dispense the multitude of samples simultaneously. Such robotic devices are commercially available, examples of manufacturers of such robotic devices include, but are not limited to Tecan, Beckmann-Coulter and Quiagen. Many robotic devices that are applied in the high-throughput screening of drug candidates can be adapted to be used with the detection devices of the present invention.

Another method of applying samples to the detection device is the use of specially engineered injection devices, as described in the examples. In this embodiment, the thermistor array and the solution to be analyzed are advantageously housed inside a chamber that may be environmentally controlled with respect to both temperature and humidity. The humidity of the atmosphere in the chamber is controlled to minimize evaporation during the thermal equilibration period, as well as, the sample transfer process. Once all components reach thermal equilibrium, the sample delivery device transfers aliquots of sample and reference solutions to the reservoirs of the thermistor array.

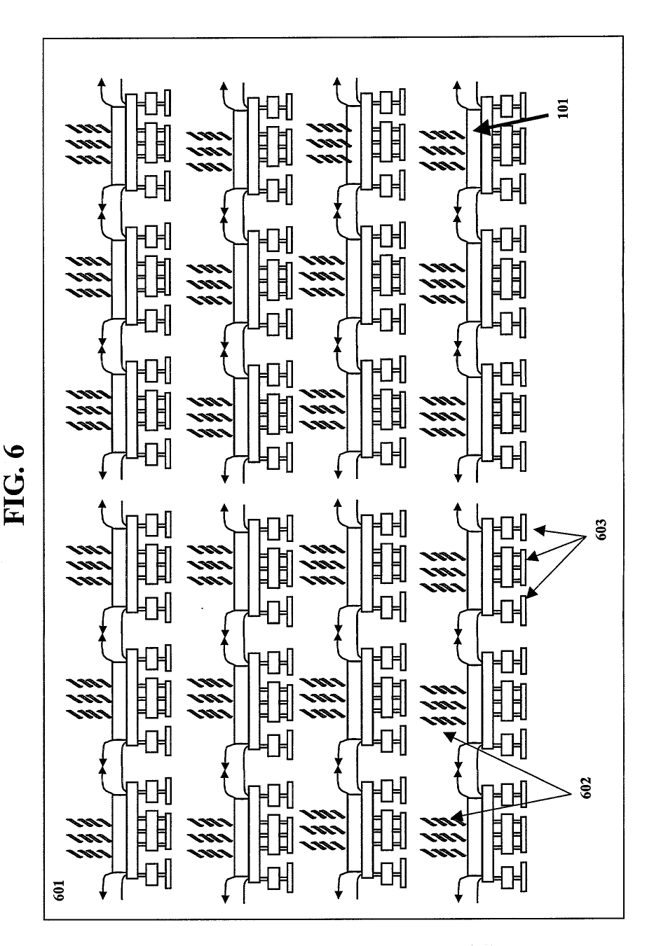

In another embodiment, the array of thermistors is arranged on a flat surface, with the probes being covalently attached to the surface. The thermistors can either be part of the surface with the probes being directly attached to them, or the thermistors could be closely associated with the surface to which the probes are covalently attached. This embodiment is exemplified in FIG. 6, which displays individually addressable thermistors 101 and accompanying electronics 603 that are incorporated into a surface 601, such as a novel type of integrated circuit. The probes 602 in this example are attached directly to the thermistors 101. As noted above, the probes associated with each thermistor may be the same molecular composition/sequence, may be different for some of the thermistors or may be different for all of the thermistors. In this embodiment, the analyte is applied on top of the surface to either all of the thermistors or to a subgroup of the thermistors.

In a particularly preferred embodiment the surface 601 is provided in the form of a microelectronic chip made from a silicon wafer or equivalent material, with probe molecules 602 of known sequence and composition attached to the surface over a measurement site. This type of assembly has several advantages. As a particular advantage, the thermistors of the detection device can be integrated in the microelectronic chip and can be manufactured by standard microelectronic chip production methods. In addition, the electronic integration on the chip can simultaneously encompass amplifier circuits and circuitry to control the measurements with respect to the time of analysis. Recent innovations in the use and layout of standard microelectronics circuits enable the fabrication of a thermal microsensor with sensitivities down to 1 μK. These innovations allow a known and thermally stable sample environment to be created, controlled and monitored throughout the process by virtue of both the physical and electronic system sensor design. Another striking advantage of such a detection device is the ability to control and minimize the size of the thermistors from millimeters to micrometers in length and width. By precisely controlling the dimensions of the thermistor material elements, the thermal mass of the sensing sites will be optimized to enhance the sensitivity and response characteristics of the device. Additionally, miniaturized thermistors allow for the use of several thousand thermistors in one detection device and therefore the simultaneous application of a very large number of thermistors in one assay. For example, thermistors with a surface area of 10 μm×10 μm can be packed on the surface of a microelectronic chip at a maximal density of 10⁶ thermistors per square centimeter.

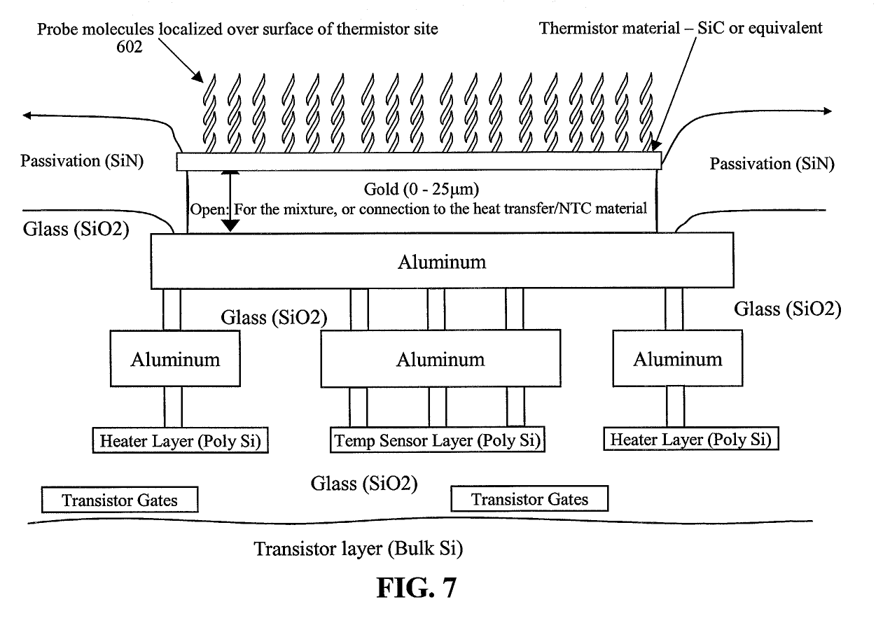

A schematic for the integration of a thermistor into a microelectronic chip is depicted in FIG. 7. With reference to FIG. 7, a thermistor material selected from silicon carbide (SiC) or any other known thermistor material is incorporated as the topmost surface of the IC system via deposition, epitaxy, physical interface/integration or any other technique known in the art. The connection between the thermistor sensing layer and the IC is made in such a manner that heat transfer is optimized vertically through the sensor layer towards the IC and heat diffusion is minimized in the lateral direction, thus optimizing the spatial resolution of signals. Finally, the surface area of the chip that is not covered by thermistor material is covered with an insulating layer of silicon nitride to avoid cross contacts of individual thermistors.

In the example depicted in FIG. 7, the probe 602 is covalently attached to the thermistor surface. The materials provided in this example, are typical of those used in the construction of integrated circuits as known to one skilled in the art. These materials are provided for the purposes of illustration only and are not to be interpreted as exclusive or limiting. In this embodiment, heating and sensing sites would be incorporated directly on the chip, permitting both extremely accurate and precise differential thermal measurements at each site of the array. Additionally, logic circuitry would be incorporated to facilitate real time signal processing, so that the reference signals could be collected simultaneously with the analyte signals, and subtracted in real time to yield a differential signal output. It is envisioned that logic could be incorporated to analyze and accept or reject the differential thermal signal based on the relative strength as compared to the reference signal, thus yielding a digital “yes or no” detection of DNA hybridization without dye molecules. In a like manner, similar logic circuitry could be incorporated to provide real time, digital indication of enzymatic activity based on the heat of the reaction. In this manner, the invention could be used to detect enzymatic amplification reactions such as, PCR or primer extension.

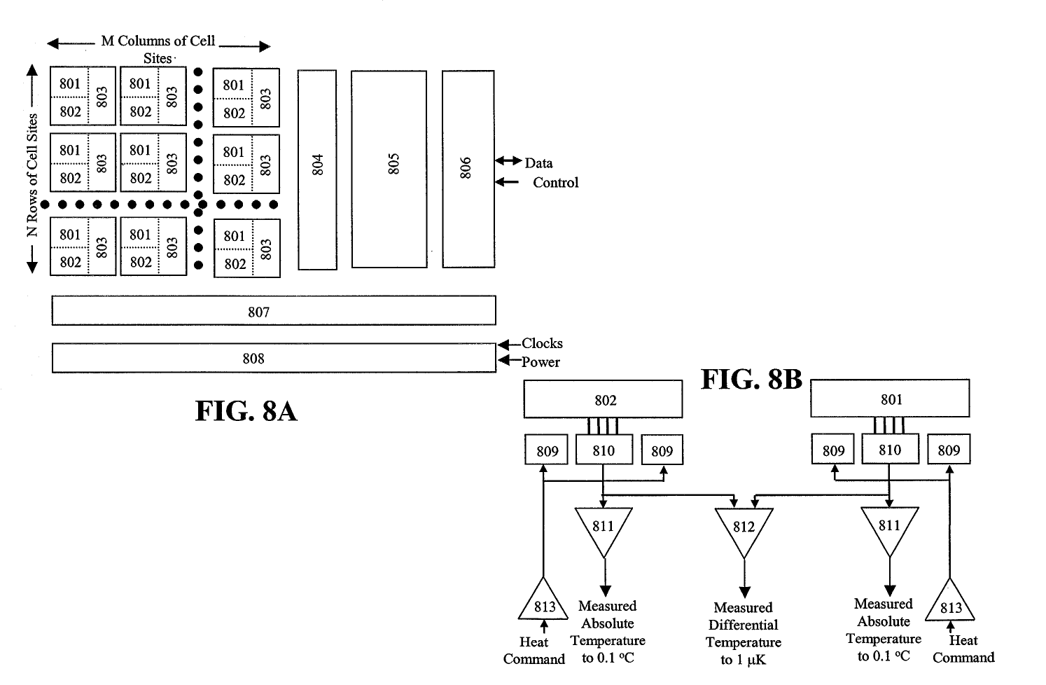

FIG. 8A illustrates an integrated circuit block diagram of the proposed system. As shown in FIG. 8A, active 802 and reference sites 801 are employed with a differential signal being acquired using control electronics 803 between the two for each measurement cell. It is envisioned that additional integrated circuitry to spatially resolve the signals from the individual cells by row and column 805, 807, as well as, communication, decision and support circuitry 806, 808 would be incorporated into said device as well. The probe molecules at both sites may be the same or different depending on the design of the experiment. The reference sites are designed such that the signals generated at these loci function as “blank” signals for comparison to the signals generated at the active sites. In one embodiment, each cell site is approximately 0.2 mm square. Each cell site may contain, for example, up to four individual active and reference cells. Up to three independent sample measurements and one control measurement are acquired for each cell site. A single 3 cm chip will provide up to 1536 cells in cell site groups of four or up to 1152 independent measurements per device. The above dimensions and numbers are given for purposes of illustration only and not to be construed as limiting to the device.

The proposed system uses a series of reference sites to validate the data acquired and multiple samples of the same experimental type are employed to assure statistical relevance and significance. FIG. 8B illustrates a measurement process block diagram showing how each reference cell 801 and active cell 802 pair is individually controlled/monitored and then differentially measured. During the temperature heating and control process, the differential amplifier 811 is used to monitor and provide control feedback for the heat applied to each cell in order to maintain a given pair of active and reference cell sites to respective temperature differentials of less than or equal to 0.1K. Temperature stability is achieved and measured by the heater driver activity 813 and the differential output 812.

In a preferred embodiment, an on-chip heating 809 and monitoring system 810 as known to one skilled in the art is employed to precisely control the temperature equilibrium of the two independent samples. When the analyte solution is exposed to a complementary known target sample localized over a sample site thermistor, a digital record of the temperature/heat of reaction response with respect to time is obtained. Since the initial temperatures can be controlled over a large range, multiple experiments over a large range of temperatures may be conducted, including but not limited to real time detection of hybridizations at more or less stringent hybridization conditions or direct measurements of DNA melting temperatures (Tm) based on the differential response of a sample site with respect to its reference site.

In this embodiment, real-time heat signals are acquired when the target and probe samples combine over a spatially discreet active sensor site 802. Through state-of-the-art low-power differential instrumentation integrated circuit design combined with electromagnetic shielded fixtures located in an isothermal environment, electric signals <10 μV corresponding to temperature resolutions of <10 μK can be accurately and precisely measured and recorded. The integrated circuit design provides self-verification and redundant measurement of the signals of interest. All data is acquired, stored, and reported in digital format suitable for subsequent statistical and other mathematical analysis.

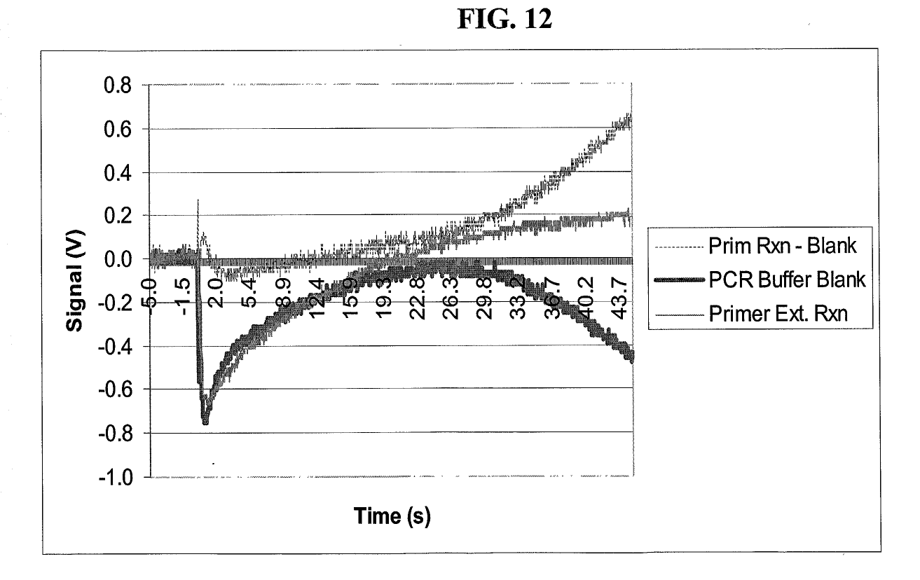

In another embodiment, enzymatic amplification reactions may be monitored in real time. The components of the enzymatic reaction may be combined over an active sensor site, and corresponding reference solutions applied over a respective reference site electronically interfaced to said sample site. Heat is added at a controlled rate via use of the on-chip heating system and subsequently allowed to cool over a defined range to initiate the reaction. The temperature may then be held isothermal or heated again at a different, but controlled rate to initiate the enzymatic amplification (extension) stage of the reaction. The system is continuously monitored during the extension stage as the independent active and reference samples react to produce differing amounts of heat. A real-time digitally sampled comparison is made between the active reaction 802 and reference samples 801 and a temperature versus time response curve for the extension stage is obtained, comparable to the one shown in FIG. 12. The difference signal is then normalized and plotted as a function of time. The direction and magnitude of this signal is then analyzed to assess the success or failure of the enzymatic reaction in real time.

In a preferred embodiment, the integrated circuit and accompanying circuitry are advantageously housed inside a chamber that is optionally environmentally controlled with respect to both temperature and humidity. Said chamber may also house a sample delivery device that comprised of an injector that delivers a single sample to the microelectronics chip. Alternatively, the delivery device may be as complex as an array of capillaries or quills mounted on a robotic device capable of controlled motion in the x, y and z planes. Said robotic sample delivery device would also be capable of expanding or contracting the array of capillaries or quills in one direction so that it could collect samples from a standard microtitre plate and deliver said samples to the test sites on the surface of the chip. This sampling device would be akin to a standard microarray printing robot as would be known to one skilled in the art. In this manner, a microtitre plate containing solutions of probe molecules that are unlabeled, as well as, reference solutions could be placed inside the chamber, and the entire system would be allowed to thermally equilibrate. The humidity of the atmosphere in the chamber would be controlled to minimize evaporation during the thermal equilibration period as well as the sample transfer process. Once all components reached thermal equilibrium, the sample delivery device would transfer aliquots of sample and reference solutions from the microtitre plate wells to the test sites on the surface of the chip for analysis.

In all embodiments of the detection devices described herein, the thermistors are interfaced to a signal processor that records the signal for a particular thermistor and relates it back to the proper x and y coordinates of the array. The individual thermistors of the array are advantageously connected with each other to build one or more arrays of bridge circuits, which allows the electronic subtraction of blank signals as the measurement proceeds. In a preferred embodiment, the detection device is interfaced with and controlled by a desktop computer. In this embodiment, the data is immediately transferred from the thermistor array upon acquisition for storage and processing. Noisy signals are optionally refined utilizing advanced signal processing algorithms including, but not limited to, Fourier Transforms or other mathematical operations as known to one skilled in the art.

The covalent attachment of the probe onto a surface that is closely associated with a thermistor or onto the surface of the thermistor itself is accomplished through techniques that are well documented in the literature ands that are currently being applied in the manufacturing of polymer supported reagents and protein or nucleic acid microarrays. Comprehensive reviews of such technologies are provided in Ramsay (1998) Nat. Biotechnol. 16:40-44, Marhall et al. (1998) Nat. Biotechnol. 16:27-31 and Schena, ed., “DNA Microarrays—A Practical Approach,” Oxford University Press 1999, each of which is incorporated herein by reference in its entirety.

Briefly, the probe is deposited on the surface either through spotting or through in situ synthesis to form a recognition layer. Methods including, but not limited to, screen printing, ink jet and microsyringe methods, as well as any other methods known to those skilled in the art are among those contemplated. The probe recognition layer that is generated by such methods is anchored either covalently or noncovalently to the surface. Standard anchoring technologies for the recognition layer, including, but not limited to silanization or disulfide bond formation, may be used as known to one skilled in the art. The recognition layer will either be deposited through spotting or synthesized in situ using ink jet or photolithographic technologies as known to one skilled in the art. Thus, a spot of a probe of known composition, sequence, and/or other properties of interest will surround and be in close proximity to each thermistor of the array. Optionally, a certain number of thermistors may remain free from coating with specific probes and the signals generated from these sites may be used as a reference to normalize the signals from the probe coated microthermistors. The heat generated when a specific binding or reaction occurs, will result in a temperature change that generates a signal increase from the microthermistor at that site as illustrated in FIGS. 1-4. Optionally, a bias may be applied across the array and the baseline signals, which are proportional to the resistances of the individual microthermistors, are recorded.

In a preferred embodiment, the probe is a nucleic acid that comprises the first member of a specific binding pair or the binding or reaction partner of an analyte and the probe is attached to the surface of the detection device by means of a covalent linkage. In one embodiment of the invention, the nucleic acid probe is attached covalently at the 3′-terminus. In another embodiment, the attachment is performed at the 5′-terminus of the nucleic acid. There is, however, no limitation to any specific mode of attaching the nucleic acid to the support and all other methods of attachment that are known to those skilled in the art are useful in the invention. These methods include, but are not limited to, ion pair formation, absorption and other non-covalent modes of attachment. Nikiforov et al., U.S. Pat. No. 5,610,287, for example, teaches nucleic acids bound to solid supports by hydrophobic interactions.

The NTC microthermistors at sites in which a specific binding or reaction has occurred generate signals through the decrease in resistance, which is proportional to the change in temperature at each individual thermistor as is characteristic of this type of thermistor. The signal processor records the signals generated and correlates the signals back to the specific location(s) within the thermistor microarray. Since the composition, sequence, and/or other properties of interest of the probe at a given site are known, the composition, sequence, and/or other properties of the second member of the specific binding pair or the analyte bound at that site may be determined.

The methods and detection devices that are provided in this invention offer a variety of advantages over the prior art. One particular advantage is that the monitoring of binding events and reactions occurs in real time as the events occur. No delay is associated with the measurement as is common with all methods that require additional steps to evaluate the generated signals. Such additional steps often include washing steps to separate bound and unbound species from each other before the signal can be evaluated. Therefore, the analysis time in detecting analytes, such as e.g. nucleic acids can be greatly reduced. A second advantage in avoiding additional manipulations is the savings in the cost of materials and labor that are associated with performing such additional steps. The cost savings incurred in this way are substantial, since the post-incubation manipulations encompass the major cost component in many assays. In addition, many post-incubation steps employ hazardous materials that can be completely avoided with the methods and devices of this invention. An example of particularly hazardous materials is the use of intercalating dyes, which are employed with common nucleic acid microarray techniques, an example being ethidium bromide, which is a carcinogenic and mutagenic substance.

Another advantage of the methods and devices of this invention is the elimination of the need for labeling the probe molecules, thus removing questions of labeling efficiency or unexpected chemical interactions of the label with the components of the assay. This also removes issues such as background fluorescence caused by fluorescent labels that can lead to high background noise. Questions of ionization efficiencies introduced with mass spectral analysis are also eliminated. Additionally, the target and probe molecules may be of the same chemical compositions only with complementary sequences, thus eliminating the need for probe specific signature molecular ions. Furthermore, since the detectors are integrated in the detection devices a direct electronic interface is provided for data storage and retrieval.

Using nucleic acids as a member of specific binding pair or as a binding or reaction partner to an analyte various types of genes can be detected by varying the nucleic acid probe employed. Useful nucleic acid probes include any probes that have base sequences complementary to any part of the base sequence of any analyte of interest including, but not limited to any of the base sequences of microorganisms contained in foods, plant viruses or viroids, pathogenic microorganisms or viruses infecting fishes, pathogenic microorganisms or viruses infecting humans and causing infectious diseases, genes causing genetic diseases, activated proto-oncogenes and minisatellite sequences. Detection devices of the invention that contain nucleic acids can be used to study and detect mutations in exons of human genes of clinical interest, including, but not limited to point mutations and deletions.

For detection devices that contain nucleic acids exemplary calculations of array parameters are shown in Tables 1, 2 and 3. These calculations indicate the heat theoretically evolved upon nucleic acid hybridizations per site on a given microthermistor array. The examples shown include theoretical enthalpies evolved upon a given hybridization. Additionally, enthalpic differences (ΔΔH) between sites for a single nucleotide polymorphism (SNP) and a splice variant comparison are shown. SantaLucia et al. (1997) Biochemistry 36:10581-10594, which is incorporated herein by reference in its entirety, have published nearest neighbor calculations that allow calculation of the heat of hybridization of an oligonucleotide. These heats of hybridization are on the order of tens of microcalories (μcal) based on the following equation:

$$\begin{matrix}Δ H {°}_{37} = n Δ {H°}_{37} ( AA / TT ) + n Δ {H°}_{37} ( A C / TG ) + \\ n Δ {H°}_{37} ( AG / TC ) + n Δ {H°}_{37} ( AT / TA ) + \\ n Δ {H°}_{37} ( CA / GT ) + n Δ {H°}_{37} ( CC / GG ) + \\ n Δ {H°}_{37} ( CG / GC ) + n Δ {H°}_{37} ( CT / GA ) + \\ n Δ {H°}_{37} ( GA / CT ) + n Δ {H°}_{37} ( GC / CG ) + \\ n Δ {H°}_{37} ( GG / CC ) + n Δ {H°}_{37} ( GT / CA ) + \\ n Δ {H°}_{37} ( TA / AT ) + n Δ {H°}_{37} ( TC / AG ) + \\ n Δ {H°}_{37} ( TG / A C ) + n Δ {H°}_{37} ( TT / AA )\end{matrix} \qquad ( Eq . 1 )$$

where n=number of this type of hybridization interaction in a given sequence. The results for a calculation of heat of reaction for a representative hybridization reaction based on Equation 1 are shown in Table 1b. Connolly & Sutherland, ibid., have demonstrated a macroarray of thermistors, not of the thin film type, that are sensitive to temperature changes in the 100 μK range. An array of thin film microthermistors with appropriate sensitivity will detect the small temperature changes resulting from numerous hybridization events occurring in the surrounding environment of the microthermistor and generate a signal for recording and processing.

In another embodiment of the present invention, the detection devices of the invention may be adapted for performing analyses based upon intermolecular affinity and/or immunochemical complex interactions. Such interactions are manifest in numerous complementary ligand/ligand receptor complexes such as antigen/antibody, antibody/anti-antibody, enzyme/enzyme receptor, hormone/hormone receptor, substrate/enzyme, drug/drug receptor, and the like. Thus, an assay may be devised in which one or the other member of the specific binding pair may be the analyte species of interest, and the other component may be used as the immobilized member of the specific binding pair. The exact assay procedure used is a choice which can be made by one skilled in the art and can be a procedure based, for example, on existing “sandwich” assays, competitive assays, or the like.

The following examples are presented for illustrative purposes only and are not intended to limit the scope of the invention.

### EXAMPLES

### Example 1.

### Detection Device from a 384-Well Plate

The listed materials and vendors used to construct the prototype device are representative only and are not intended to be an exclusive list. Equivalent equipment and materials from different suppliers utilized in a similar manner as known to one skilled in the art can be applied alternatively.

A prototype biothermal hybridization device (sensor plate) 500 (FIG. 5A) was constructed by modifying a standard 384-well microtitre plate (Model #242765 Nalge Nunc International, Rochester, N.Y.) to incorporate 10 MΩ hermetically sealed negative temperature coefficient (NTC) thermistors 504 (FIG. 5B) (Part #QT06002-133-13, Quality Thermistors, Boise, Id.). Holes were drilled in the bottoms of 16 wells to diameters slightly smaller than the 0.042″ diameter of the NTC thermistors 504. Arrays of four thermistors (2×2 pattern) 502 were then incorporated into the modified plate to create four arrays with four members each 502.1, 502.2, 502.3, 502.4. FIG. 5B illustrates an expanded view of 1 array. With reference to FIG. 5B, each NTC thermistor 504 was sealed into the bottom of the individual well of the array using epoxy. Using the leads 505 from the thermistors the individual NTC elements of each array were wired together 505 to form a standard bridge circuit as illustrated in FIG. 5C. The four bridge circuits were then interfaced to a control board with standard OPAMP electronics 506, including amplification (gain) settings of 10, 100 and 1000. The measured, amplified signal was collected as the output 508 from the bridge circuit and stored in the PC. All data reported were acquired using a gain of 100.

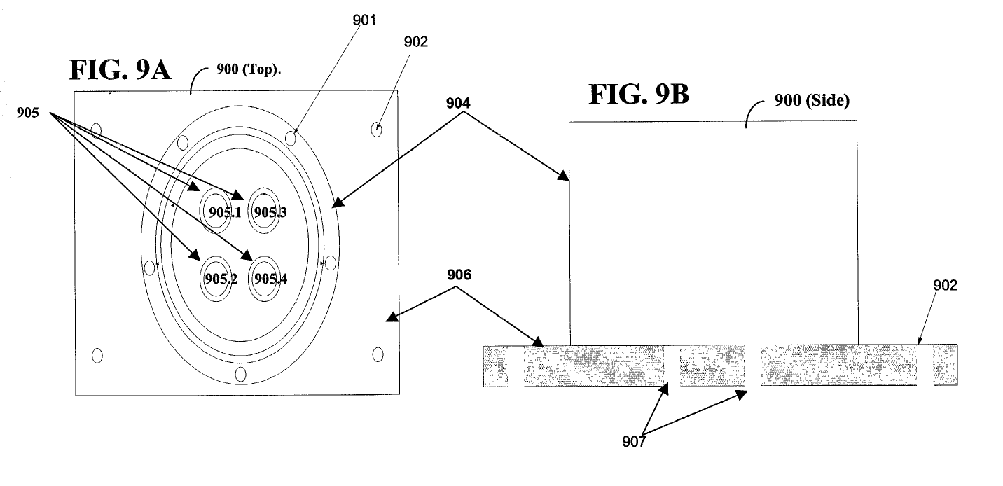

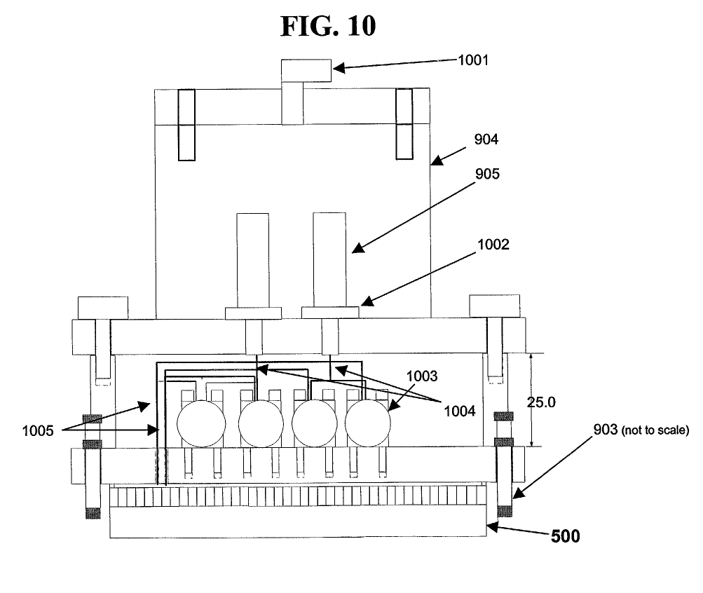

An injector 900 was constructed using LFVA microdispense valves 1003, 062 minstac Teflon tubing 1004, 1005 (The Lee Company, Essex, Conn.), and Swagelock components 905 (Houston Valve & Fitting Co, Houston, Tex.) as illustrated in FIGS. 9 and 10. The Swagelock components were assembled into four reservoirs 905.1, 905.2, 905.3 and 905.4 and housed inside a sealed argon chamber 904 with a removable lid. The argon chamber 904 was mounted on a base 906. Connections between the different sections of the injector (reservoirs to valves, valves through base to nozzles, injector to plate stand 903) were accomplished using threaded holes 901, 902 and 907. The valves were plumbed between the Swagelock reservoirs and the injector nozzles using the Lee tubing and additional Swagelock fittings 1002, and were controlled using a programmable logic controller (PLC) (Model #T100MD1616+, Triangle Research, Inc., San Jose, Calif.) interfaced to a desktop PC (Dell Computer, Austin, Tex.). Data were collected using a digital oscilloscope (Model #HP54600B, Agilent Technologies, Palo Alto, Calif.) interfaced to the same PC. Injections were delivered using positive Ar pressure regulated to 5 psig. Ar was introduced into the Ar chamber via the Ar inlet valve 1001.

The reservoirs were loaded with buffer or analyte to be analyzed, and positioned over the sensor plate 500 as shown in FIG. 10. The principle behind this injector configuration was to have the ability to inject an analyte sample and reference sample simultaneously to standardize injections and minimize artifacts. Injection volumes were calibrated by correlating injection times with delivery volume at the given Ar pressure. Once the calibration volume was determined, all injection times were stored in the PLC program for that volume/injection profile. To ensure thermal equilibrium and minimize thermal artifacts, the empty space surrounding the reservoirs within the Ar chamber and the base of the thermal sensing prototype device were immersed in Dowtherm 200 silicon heat transfer oil (Dow Corning, Midland, Mich.). The entire assembly was placed inside an oven and equilibrated at either 37° C. (DNA/DNA hybridization experiments) or 50° C. (primer extension experiments).

The described apparatus was constructed on a larger scale than is anticipated for the preferred embodiment of the detection device utilizing microelectronic chips, but the design principles of the electronics and environmental controls are anticipated to transfer easily to a much smaller integrated circuit (IC or chip) configuration. Significant gains in sensitivity and analysis speed are anticipated once the device is migrated to the IC configuration, as the thermal mass of the sensor that must be excited by the reactions will be greatly reduced by manufacturing the sensor as a thin film overlaying the solid support and electronics.

### Example 2.

### Measurement of Nucleic Acid Hybridization

### Materials

Tm buffer: 1 M sodium chloride (NaCl), 10 mM sodium cacodylate (C₂H₆As*NaO₂*3H₂O), 0.5 mM EDTA, all dissolved in 18 MΩ H₂O at pH 7.

DNA oligonucleotides were all synthesized in house at Proligo, LLC and purified to a minimum purity of 95% using anion exchange high pressure liquid chromatography (HPLC). Final molecular masses and quantities were determined using a MALDI MS and a UV-Vis spectrophotometer, respectively. Sequence data is listed in Table 4.

PCR buffer: 7.4 mM MgCl₂, 29.7 mM Tris*HCl in 18 MΩ H₂O.

Master Mix: 0.3 μM primer DNA, 0.3 μM template DNA, 100 μM dNTPs, 0.01 U pyrophosphatase/μL.

Thermosequenase solution: 0.08 U thermosequenase/μL in PCR buffer.

### Methods

To perform the experiments, the reservoirs of the injector were filled with either Ar (for Ar blank injections), Tm Buffer (for buffer blank injections), DNA solution (oligonucleotides diluted in Tm buffer) or thermosequenase solution (for primer extension experiments). The wells in the sensor plate beneath the injector nozzles were filled with 50 μL of the respective test solutions, such as Tm buffer for blank injections, complementary oligonucleotide solutions for DNA hybridization tests or Master Mix for primer extension reactions. After a minimum equilibration time of 20-25 minutes, and once a stable baseline was observed using the digital oscilloscope, the injection was performed.

Ar Blank Injections—Electronic Noise Determination

Initially, “blank” injections of Ar only were performed to determine the baseline electronic noise of the device. To perform these injections the device assembly was placed into the oven and allowed to thermally equilibrate. Equilibration was evaluated based on the stability of the electronic baseline observed on the digital oscilloscope. To perform the test, the PLC actuated the valves, allowing Ar to flow for a few milliseconds into the thermal sensor prototype. The digital oscilloscope was interfaced to the PLC so that when the PLC actuated the valves, the oscilloscope was triggered and a baseline was captured. The data was then transferred to the PC. The electronic noise level was determined to be <100 μV based on the results.

Buffer Blank Injections—Chemical Noise Determination

Buffer blank injections were performed by filling the reservoirs of the injector with 200 μL of Tm buffer, and positioning the injector such that the nozzles were positioned over one leg of a bridge circuit within an array, i.e. reservoirs 2 and 4. The corresponding wells within the array were filled with 50 μL of the same Tm buffer. The injector and sensor plate prototype were allowed to thermally equilibrate in the oven as noted above.

After thermal equilibration of the entire assembly, 6.5 μL of the buffer from each of the reservoirs was injected into the respective wells of the sensor prototype, and the thermal signature was captured. The subsequent volumes within the respective wells were measured using Eppendorf pipettes, and the data was only accepted when the volume difference between the two wells was <4 μL. This criterion was chosen to minimize the effects of injection artifacts. Typical post injection well volume differences were closer to 1 μL.

DNA/DNA Hybridization Experiments

The DNA hybridization experiments were accomplished in a like manner to the buffer blank measurements, the differences being that the respective wells were filled with 50 μL of complementary DNA sequence. Reservoir #2 was filled with DNA analyte solution, nominally 20 times more concentrated than the DNA solution in the well plate and reservoir #4 was filled with Tm buffer. Analyte (6.5 μL) was injected into one leg of the bridge, and simultaneously, the same volume of Tm buffer was injected into the opposite leg. This served to reference the signal to the blank, and accentuate the differences in heats of reaction between the two legs of the bridge circuit. Data was captured on the digital oscilloscope and transferred to the PC. DNA injections were performed until three samples were acquired with a volume difference between wells that met the same rejection criteria applied to the blank injections.

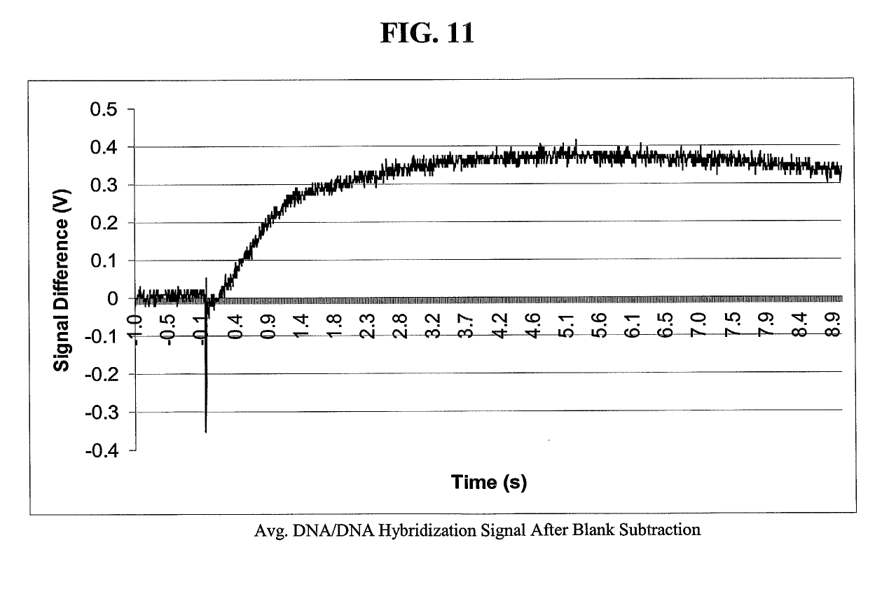

The data were analyzed by subtracting corresponding blank measurements from DNA measurements, (i.e. DNA Inj. #1−Blank Inj. #1), and plotting the resultant signal differences versus time. Sets of data for DNA/DNA hybridizations were collected on two separate days and corrected with respect to blank signals as described above. DNA signal differences were compared to corresponding buffer blank differences using the Student's t statistical test, assuming unequal variances. Comparison of the respective t values and t critical values, as known to one skilled in the art, indicated that the DNA data were consistently distinguishable from the blank with 95% confidence. A representative averaged plot of the DNA−blank signal difference and the results of the tests are shown in FIG. 11 and Table 5, respectively. The results of these tests indicate that the DNA to DNA hybridization experiments produce a consistent signal within groups that is distinguishable from the signal produced from blank injections. This clearly demonstrates non-dye, thermal detection of DNA hybridization using the NTC thermistors disclosed herein.

Primer Extension Experiments

Primer extension reactions were performed in a like manner to the DNA hybridization reactions. Short (“primer”) and long (“template”) oligonucleotide sequences were diluted in PCR buffer along with pyrophosphatase and dinucleotide triphosphates (dNTPs) corresponding to each of the four nucleobases to form the Master Mix. Master Mix (50 μL) was loaded into wells #2 and #4. PCR buffer (200 μL) was loaded into reservoirs 2 and 4 of the injector, and after thermal equilibration, the of PCR buffer (6.5 μL) was injected into both wells and the electrical signal was collected using the digital oscilloscope and monitoring the reaction for 50 seconds. This constituted the blank experiment. Blank experiments were repeated until 3 sets of data that met the volume rejection criteria were acquired. The actual primer extension experiments were accomplished in the same manner as the blank experiments. The only difference being that reservoir #2 of the injector was loaded with thermosequenase solution (200 μL of 0.08 U/μL). Reservoir #4 remained filled with PCR buffer (200 μL). After standard thermal equilibration, 6.5 μL was injected from each reservoir into the respective wells of the sensor plate to initiate the primer extension reaction. This corresponded to injecting 0.5 U of thermosequenase into well #2. For a positive result, the oligonucleotides had to hybridize in the Master Mix prior to injection of the thermosequenase. The thermosequenase enzyme would extend the hybridized oligonucleotide sequences in the Master Mix solution in a manner as is known to one skilled in the art. The pyrophosphatase enzyme would consume the phosphate byproducts from the incorporation of the dNTPs and release heat as a byproduct, thus acting to amplify the thermal signal. The signal was again captured on the digital oscilloscope for 50 seconds and transferred to the PC. The blank signal was subtracted from the primer extension reaction signal and the resulting voltage difference was plotted versus time as with the DNA hybridization. The results are set forth in FIG. 12.

As seen in FIG. 12, and born out by the Student's t analysis, a strong, statistically relevant signal, begins approximately 15 seconds into the experiment. This is shown by the divergence between the blank and primer extension reaction signals, plotted as (Prim. Rxn−Blank). The delay in the reaction initiation is assumed to be due to limiting enzyme conditions under which the experiment was conducted. Enzyme concentration of 0.5 U vs. a standard concentration of 8 U was used to ensure that a signal would be generated in a measurable time frame for the prototype and that the signal would not be missed due to too rapid reaction kinetics. This data clearly demonstrates the ability of the biosensor prototype with the NTC thermistors to measure the heat of an enzymatic reaction without utilizing dyes. It is envisioned that such a device, incorporated with the appropriate integrated circuitry and logic, could be utilized as a real time monitor for such enzymatic reactions as primer extension, mismatch repair, or real time polymerase chain reaction, eliminating the need for dye incorporation or utilization.

DNA Mismatch Experiments

DNA mismatch studies were performed in the same manner as the above mentioned DNA hybridization studies, with the goal of determining if matched and mismatched DNA could be distinguished based on the heats released upon the respective hybridization events. Blank signals were collected in the same manner as previously noted. The analyte DNA (200 μL) was loaded in reservoir #2 and Tm buffer (200 μL) was loaded in reservoir #4. For the match study, 50 μL of perfectly complementary DNA solution was loaded in to wells #2 and #4 of the array. An injection was performed after thermally equilibrating the system in the manner previously noted. After three injections of perfectly complementary DNA experiments that met the volume rejection criteria were collected, the DNA solutions in the wells of the sensor plate were removed, the wells rinsed with 18 MΩ H₂O and Tm buffer, and a different oligonucleotide solution of the same concentration was loaded in to wells 2 and 4. The oligonucleotide solutions loaded in the wells for these experiments contained a single mismatch (non-complementary base) with respect to the analyte DNA solution in the middle of the sequence. Experiments were performed in the same manner as previously described. FIG. 13 illustrates averaged, representative results from these experiments, showing the signals acquired from the perfect match, single DNA base mismatch, and blank experiments. Examination of the end point of the graph, indicating the heat produced by the hybridization experiments over the course of the experiment, clearly demonstrates that the signal generated by the perfectly matched oligo pair is higher (wanner) than the signal of the singly mismatched oligo pair. Furthermore, the signal from the singly mismatched oligo experiment more closely resembles that of the blank injection than the perfect match. This data indicates that the thermal detection technique is able to distinguish between perfectly matched and mismatched DNA sequences.

TABLE 1a
Calculation of Array Parameters

| | |
|---|---|
| Area of microarray spot (1000 μm diameter) (μm²) = | 785397.5 |
| Area of thermistor in spot (50 μm × 50 μm) (μm²) = | 2500 |
| Area for target attachment (μm²) | 782897.5 |
| (assuming no direct attachment to thermistor) = | |
| Volume per spot for hybridization (μm³) | 78289750.0 |
| (assuming 100 μm height of hybridization chamber) = | |
| Convert volume to μL (assuming 1 cc = 1 mL) = | 0.0783 |
| Concentration of spotting solution (ng/μL) = | 1000 |
| Avg. MW of 20mer DNA (daltons) = | 6440 |
| nmol/μL (average) = | 0.155 |
| pmol/microarray spot (average) = | 12.16 |

TABLE 1b
Calculation of Heat of Reaction for a Representative
Hybridization Reaction Based on Equation 1

| Target DNA Sequence | Probe DNA Sequence | ΔH NN (kcal/mol) |
|---|---|---|
| C | G | −8.5 |
| A | T | −8.2 |
| G | C | −8.4 |
| T | A | −7.8 |
| C | G | −8.5 |
| A | T | −8.2 |
| G | C | −8.4 |
| T | A | −7.8 |
| C | G | −8.5 |
| A | T | −8.2 |
| G | C | −8.4 |
| T | A | −7.8 |
| C | G | −8.5 |
| A | T | −8.2 |
| G | C | −8.4 |
| T | A | −7.8 |
| C | G | −8.5 |
| A | T | −8.2 |
| G | C | −8.4 |
| T | A | −7.8 |
| | Total | −164.5 |

| | |
|---|---|
| initiation w/terminal G-C | 0.1 |
| ΔH for sequence (kcal/mol) | −164.4 |
| ΔH for sequence (cal/mol) | −164400 |
| heat evolved from pairing per spot (μcal) | −2.00 |

TABLE 2a
Calculation of Array Parameters for SNP Example

| | |
|---|---|
| Diameter of microarray spot (μm) = | 500 |
| Area of microarray spot (μm²) = | 196349.375 |
| Diameter of thermistor = | 100 |
| Area of thermistor in spot (50 μm × 50 μm) (μm²) = | 10000 |
| Area for target attachment (μm²) | 186349.375 |
| (assuming no direct attachment to thermistor) = | |
| Volume per spot for hybridization (μm³) | 18634937.5 |
| (assuming 100 μm height of hybridization chamber) = | |
| Convert volume to μL = | 0.0186 |
| Concentration of spotting solution (ng/μL) = | 1000 |
| Avg. molec wt of 20mer NA oligonucleotide = | 6440 |
| Avg. # of nmol per μL = | 1.55 |
| Avg # of pmol per microarray spot = | 28.94 |

TABLE 2b
Calculation of Heat of Reaction for SNP Example

| Target DNA Sequence | Wildtype | |
|---|---|---|
| (Wildtype complement) | Probe DNA Sequence | ΔH NN (kcal/mol) |
| G | C | −8.4 |
| T | A | −7.8 |
| C | G | −8.5 |
| G | C | −8.4 |
| G | C | −8.4 |
| A | T | −8.2 |
| G | C | −8.4 |
| A | T | −8.2 |
| C | G | −8.5 |
| C | G | −8.5 |
| A | T | −8.2 |
| T | A | −7.8 |
| T | A | −7.8 |
| C | G | −8.5 |
| C | G | −8.5 |
| C | G | −8.5 |
| A | T | −8.2 |
| A | T | −8.2 |
| A | T | −8.2 |
| A | T | −8.2 |

| | |
|---|---|
| 5′ terminal TA basepair penalty | 0.4 |
| ΔH for sequence (kcal/mol) | −165 |
| ΔH for sequence (cal/mol) | −165000 |
| heat evolved from pairing per spot (μcal) | −4.77 |

TABLE 2c
Calculation of ΔΔH for SNP Example

| Target DNA Sequence | Mutant | |
|---|---|---|
| (exon 7 & 8 (wildtype)) | Probe DNA Sequence | ΔH NN (cal/mol) |
| G | C | −8.4 |
| T | A | −7.8 |
| C | G | −8.5 |
| G | C | −8.4 |
| G | C | −8.4 |
| A | T | −8.2 |
| G | C | −8.4 |
| A | T | −8.2 |
| C | G | −8.5 |
| C | C | 0 |
| A | T | −8.2 |
| T | A | −7.8 |
| T | A | −7.8 |
| C | G | −8.5 |
| C | G | −8.5 |
| C | G | −8.5 |
| A | T | −8.2 |
| A | T | −8.2 |
| A | T | −8.2 |
| A | T | −8.2 |

| | |
|---|---|
| initiation w/terminal G-C | 0.4 |
| ΔH for sequence (kcal/mol) | −156.5 |
| ΔH for sequence (cal/mol) | −156500 |
| heat evolved from pairing per spot (μcal) | −4.53 |
| ΔΔH Wildtype compared to Mutant (μcal/spot) | 0.25 |

TABLE 3a
Calculation of Array Parameters for Splice Variant Example

| | |
|---|---|
| Diameter of microarray spot (μm) = | 500 |
| Area of microarray spot (μm²) = | 196349.375 |
| Diameter of thermistor = | 100 |
| Area of thermistor in spot (50 μm × 50 μm) (μm²) = | 10000 |
| Area for target attachment (μm²) | 186349.375 |
| (assuming no direct attachment to thermistor) = | |
| Volume per spot for hybridization (μm³) | 18634937.5 |
| (assuming 100 μm height of hybridization chamber) = | |
| Convert volume to μL = | 0.0186 |
| Concentration of spotting solution (ng/μL) = | 1000 |
| Avg. molec wt of 20mer NA oligonucleotide = | 6440 |
| Avg. # of nmol per μL = | 1.55 |
| Avg # of pmol per microarray spot = | 28.94 |

TABLE 3b
Calculation of Heat of Reaction for Splice Variant Example

| Target DNA Sequence | Wildtype | |
|---|---|---|
| (exon 7 & 8 (wildtype)) | Probe DNA Sequence | ΔH NN (kcal/mol) |
| A | T | −8.2 |
| G | C | −8.4 |
| G | C | −8.4 |
| A | T | −8.2 |
| C | G | −8.5 |
| C | G | −8.5 |
| C | G | −8.5 |
| C | G | −8.5 |
| T | A | −7.8 |
| G | C | −8.4 |
| G | C | −8.4 |
| T | A | −7.8 |
| C | G | −8.5 |
| C | G | −8.5 |
| A | T | −8.2 |
| T | A | −7.8 |
| A | T | −8.2 |
| G | C | −8.4 |
| G | C | −8.4 |
| G | C | −8.4 |
| A | T | −8.2 |
| C | G | −8.5 |
| C | G | −8.5 |
| T | A | −7.8 |
| A | T | −8.4 |
| A | T | −8.4 |
| T | A | −7.8 |

| | |
|---|---|
| 5′ terminal TA basepair penalty | 0.4 |
| ΔH for sequence (kcal/mol) | −223.2 |
| ΔH for sequence (cal/mol) | −223200 |
| heat evolved from pairing per spot (μcal) | −6.46 |

TABLE 3c
Calculation of ΔΔH for Splice Variant Example

| Target DNA Sequence | Mutant | |
|---|---|---|
| (exon 7 & 8 (wildtype)) | Probe DNA Sequence | ΔH NN (cal/mol) |
| A | T | −8.2 |
| G | C | −8.4 |
| G | C | −8.4 |
| A | T | −8.2 |
| C | G | −8.5 |
| C | G | −8.5 |
| C | G | −8.5 |
| C | G | −8.5 |
| T | A | −7.8 |
| G | C | −8.4 |
| G | C | −8.4 |
| T | A | −7.8 |
| C | G | −8.5 |

| | |
|---|---|
| initiation w/terminal G-C | 0.4 |
| ΔH for sequence (kcal/mol) | −107.7 |
| ΔH for sequence (cal/mol) | −107700 |
| heat evolved from pairing per spot (μcal) | −3.12 |
| ΔΔH Wildtype compared to Mutant (μcal/spot) | −3.34 |

TABLE 4
DNA Sequences tested in Prototype Experiments

| | | | | |
|---|---|---|---|---|
| | | | Concentration | SEQ ID |
| Name | Sequence | Length | (μM) | NO: |

| | | | | |
|---|---|---|---|---|
| (CA)₁₀ | CACACACACACACACACACA | 20 | 250 | 1 |
| (TG)₁₀ | TGTGTGTGTGTGTGTGTGTG | 20 | 10700 | 2 |
| Primer Oligo | GCTGCCGGGAGGCTATCAA | 19 | 0.3 | 3 |
| Template Oligo | TACAAACTCATAGGCGATCCT | 41 | 0.3 | 4 |
| | TTTGATAGCCTCCCGGCAGC | | | |

TABLE 5
Summary of t-Test Statistics for DNA vs. Blank Injections*

| | Normalized | | |
|---|---|---|---|
| | DNA − Blank | Normalized | t-Statistic |
| Injection | Signal | Blank | (t-Critical, |
| # | Difference Mean | Signal Mean | 2 tail = 1.96) |
| 2 | 0.3689 | −0.2370 | 155.65 |
| 3 | 0.3188 | −0.1946 | 125.45 |
| 4 | 0.1715 | −0.3225 | 106.65 |
| Average | 0.2864 | −0.2514 | −140.28 |

*All tests were conducted at 95% confidence level, assuming 0 difference in means.

1. A method for detecting binding between a first member of a specific binding pair and a second member of a specific binding pair, said method comprising:
a) providing a detection device, wherein said detection device comprises an array of spatially addressable thermistors;
b) providing the first member of the specific binding pair, wherein said first member of the specific binding pair is closely associated with a spatially addressable thermistor in the detection device;
c) contacting the detection device with a sample containing one or more of the second member of the specific binding pair; and
d) detecting the binding between said binding pair members via thermal analysis.

2. The method of claim 1 wherein said binding pair is selected from the group consisting of complementary nucleic acids, antibody/antigen, ligand/receptor, enzyme/substrate and aptamer/target.

3. The method of claim 2 wherein the specific binding pair comprises chimeric molecules.

4. The method of claim 2 wherein said complementary nucleic acids are selected from the group consisting of DNA/DNA, DNA/RNA, DNA/LNA, DNA/siRNA and DNA/PNA.

5. The method of claim 2 wherein said binding is hybridization between the complementary nucleic acids.

6. The method of claim 1 wherein the first member of the specific binding pair is attached to the detection device via a covalent bond or a non-covalent bond.

7. The method of claim 1 wherein said first member of the specific binding pair is localized over the detection device in the form of a solution that is in close proximity to the thermistor of the detection device.

8. The method of claim 1 wherein said thermistor is a negative temperature coefficient (NTC) thermistor.

9. The method of claim 1 wherein each member of said binding pair contains a reactive moiety.

10. The method of claim 9 wherein said reactive moiety is selected from groups selected to maximize the heat of reaction.

11. The method of claim 9 wherein and said binding between said binding pair members is a covalent reaction between the reactive moieties.

12. The method of claim 9 wherein said binding between said binding pair members is via noncovalent interactions followed by covalent reaction between said reactive moieties on each member of said binding pair.

13. The method of claim 1 wherein the detection device provides a real time, digital profile of the binding between said binding pair members as is occurs in the detection device.

14. A method for detecting an analyte in a solution said method comprising:
a) providing a detection device, wherein said detection device is comprised of an array of spatially addressable thermistors;
b) providing a binding or reaction partner to said analyte, wherein said binding or reaction partner to the analyte is closely associated with a spatially addressable thermistor in the detection device;
c) contacting the detection device with a sample containing one or more analytes; and
d) detecting the binding or reaction between said analyte and its binding or reaction partner by thermal analysis.

15. The method of claim 14 wherein the binding or reaction partner and the analyte are selected from the group consisting of complementary nucleic acids, antibody/antigen, ligand/receptor, enzyme/substrate and aptamer/target.

16. The method of claim 15 wherein the binding or reaction partner and the analyte are comprised of chimeric molecules.

17. The method of claim 15 wherein said complementary nucleic acids are selected from the group consisting of DNA/DNA, DNA/RNA, DNA/LNA, DNA/siRNA and DNA PNA.

18. The method of claim 15 wherein said binding is hybridization between the complementary nucleic acids.

19. The method of claim 14 wherein the binding or reaction partner is attached to the detection device via a covalent bond or a non-covalent bond.

20. The method of claim 14 wherein said the binding or reaction partner is localized over the detection device in the form of a solution that is in close proximity to the thermistors of the detection device.

21. The method of claim 14 wherein said thermistors are negative temperature coefficient (NTC) thermistors.

22. The method of claim 14 wherein the binding or reaction partner and the analyte each contain a reactive moiety.

23. The method of claim 22 wherein said reactive moiety is selected from groups selected to maximize the heat of reaction.

24. The method of claim 22 wherein and said reaction between the analyte and its binding or reaction partner is a covalent reaction between the reactive moieties.

25. The method of claim 22 wherein said reaction between the analyte and its binding or reaction partner is via noncovalent interactions followed by covalent reaction between said reactive moieties.

26. The method of claim 14 wherein two molecules jointly provide the binding or reaction partner of an analyte and wherein at least one of the two binding or reactions is closely associated with the thermistors of the detection device.

27. The method of claim 26 wherein at least one of the two molecules and the analyte each contain a reactive moiety.

28. The method of claim 27 wherein said reaction is a chemical ligation.

29. The method of claim 14 wherein the binding between the analyte and its binding partner is used to distinguish between perfectly complementary sequences and non-complementary sequences in which the non-complementary elements may comprise one or more elements of the mismatched sequence.

30. The method of claim 14 wherein the binding between the analyte and its binding partner comprises part of an enzymatic amplification reaction.

31. The method of claim 30 wherein said enzymatic amplification is a polymerase chain reaction or a primer extension reaction.

32. The method of claim 14 wherein the detection device provides a real time, digital profile of the binding or reaction between the analyte and its binding or reaction partner.

33 A detection device comprised of an array of addressable thermistors, wherein each of said addressable thermistors is closely associated to either a first member of a specific binding pair or to a binding or reaction partner to an analyte.

34. The detection device of claim 33 wherein said thermistors are negative temperature coefficient (NTC) thermistors.

35. The detection device of claim 33 wherein the array of addressable thermistors is further comprised of reservoirs that encompass the thermistors.

36. The detection device of claim 35 wherein said reservoir is a multi-well microtitre plate.

37. The detection device of claim 33 wherein the array of addressable thermistors is integrated onto a planar surface.

38. The detection device of claim 37 wherein said planar surface is a microelectronic chip.

39. The detection device of claim 38 wherein said microelectronic chip is a silicon wafer or equivalent material thereof.

40. The detection device of claim 33 wherein the means of association of the binding pair or reaction partners with the thermistor is selected from covalent or non-covalent attachment, or spatial localization in the form of a solution that is in close proximity to the thermistors of the detection device.

41. The detection device of claim 33 wherein said array of thermistors is housed in a chamber to control both temperature and humidity.

42. The detection device of claim 33 wherein each of said thermistors is interfaced to a processing unit.

43. The detection device of claim 33 further comprising means to provide a real time signal output.

44. A method for preparing a detection device comprising:
a) providing a solid support; and
b) associating at least two thermistors with the solid support in an addressable array, connecting the thermistors and further associating at least one spot of a first member of a specific binding pair or reaction partner of known composition surrounding and in close proximity to the thermistors, wherein each thermistor is connected to a signal processor.

45. The method of claim 44 wherein said thermistors are negative temperature coefficient (NTC) thermistors.

46. The method of claim 44 wherein said solid support is selected from the group consisting of Langmuir-Bodgett films, functionalized glass, germanium, silicon, silicon carbide, PTFE, polystyrene, gallium arsenide, gold and silver.

47. The method of claim 44 wherein the array of addressable thermistors is further comprised of reservoirs that encompass the thermistors.

48. The method of claim 47 wherein said reservoir is a multi-well microtitre plate.

49. The method of claim 44 wherein the array of addressable thermistors is integrated onto a planar surface or a spherical surface.

50. The method of claim 49 wherein said planar surface is a microelectronic chip.

51. The method of claim 50 wherein said microelectronic chip is a silicon wafer.

52. The method 44 wherein the means of association of the binding pair or reaction partners with the thermistor is selected from a covalent or non-covalent attachment, or sapatial localization in the form of a solution that is in close proximity to the thermistors of the detection device.

53. The method of claim 44 wherein said array of thermistors is housed in a chamber to control both temperature and humidity.

54. The method of claim 44 wherein each of said thermistors is interfaced to a processing unit.

55. The method of claim 44 further comprising providing means to produce a real time signal output.

56. The method of claim 44 wherein said thermistors are connected via a bridged circuit configuration

57. An instrument for detecting a thermal event comprising:
a) a detection device comprised of an array of addressable thermistors, wherein each of said addressable thermistors is closely associated to either a first member of a specific binding pair or to a binding or reaction partner to an analyte and wherein each of said thermistors is interfaced to a processing unit; and
b) a delivery device comprised of an injector, for introducing a substance to the detection device.

58. The instrument of claim 57 further comprising an environmental control chamber to house the delivery device and detection device.

59. The instrument of claim 57 wherein said delivery device is an array of capillaries, quills or microdispensing nozzles mounted on a robotic device capable of controlled motion in the x, y and z direction.
